(19) United States
(12) Patent Application Publication
(10) Pub. No.: US 2022/0213517 A1
(43) Pub. Date: Jul. 7, 2022

(54) PRODUCTION OF POLYHYDROXYBUTYRATE IN WOOD-LJUNGDAHL MICROORGANISMS

(71) Applicant: LanzaTech, Inc., Skokie, IL (US)

(72) Inventors: Ryan Christopher Tappel, Skokie, IL (US); James Bruce Yarnton Haycock Behrendorff, Frederiksberg (DK); Michael Koepke, Chicago, IL (US); Esteban Marcellin, St. Lucia (AU); Renato de Souza Pinto Lemgruber, St. Lucia (AU); Kaspar Valgepea, St. Lucia (AU); Lars Nielsen, St. Lucia (AU)

(21) Appl. No.: 17/656,624

(22) Filed: Mar. 25, 2022

Related U.S. Application Data
(63) Continuation-in-part of application No. 16/152,354, filed on Oct. 4, 2018, now Pat. No. 11,306,333.
(60) Provisional application No. 62/568,127, filed on Oct. 4, 2017.

(51) Int. Cl. C12P 7/625; C12N 9/04; C12N 9/10; C12N 1/20

(52) U.S. Cl. CPC C12P 7/625; C12N 9/0006; C12N 9/1029; C12R 2001/145; C12Y 101/01036; C12Y 101/01157; C12Y 203/01009; C12N 1/20

(57) ABSTRACT

The invention provides microorganisms and methods for the production of polyhydroxybutyrate (PHB) from gaseous substrates. In particular, the invention provides a non-naturally occurring Wood-Ljungdahl microorganism comprising (a) an enzyme that converts acetyl-CoA to acetoacetyl-CoA, (b) an enzyme that converts acetoacetyl-CoA to 3-hydroxybutyryl-CoA, and (c) an enzyme that converts 3-hydroxybutyryl-CoA to polyhydroxybutyrate, and methods related thereto.

### CROSS REFERENCE TO RELATED APPLICATIONS

This application is a continuation-in-part of U.S. application Ser. No. 16/152,354 filed Oct. 4, 2018, which claims the benefit of U.S. Patent Application 62/568,127 filed Oct. 4, 2017, the entirety of which is incorporated herein by reference.

### FIELD OF THE INVENTION

The present invention relates to genetically engineered microorganisms and methods for the production of polyhydroxybutyrate (PHB) by microbial fermentation, particularly by microbial fermentation of a gaseous substrate.

### BACKGROUND OF THE INVENTION

Petroleum-derived plastics have become essential to modern life, largely due to their lightness, robustness, durability, and resistance to degradation. However, dependence on petroleum-derived plastics has resulted in a score of serious problems, including crude oil depletion, pollution, and landfill accumulation. To decrease the environmental impacts of plastics, efforts are underway to replace conventional petroleum-derived polymers with biopolymers such as polylactide, polysaccharides, aliphatic polyesters and polyhydroxyalkanoates that possess similar physicochemical properties as conventional plastics (Anjum, Int J Biol Macromol, 89: 161-174, 2016). However, microorganisms and methods for producing such biopolymers are still largely undeveloped.

### SUMMARY OF THE INVENTION

The invention provides a genetically engineered microorganism capable of producing PHB. In particular, the invention provides a non-naturally occurring Wood-Ljungdahl microorganism comprising (a) an enzyme that converts acetyl-CoA to acetoacetyl-CoA, (b) an enzyme that converts acetoacetyl-CoA to 3-hydroxybutyryl-CoA, and (c) an enzyme that converts 3-hydroxybutyryl-CoA to PHB.

In one embodiment, the enzyme that converts acetyl-CoA to acetoacetyl-CoA is an acetyl-CoA C-acetyltransferase (EC 2.3.1.9). For example, the acetyl-CoA C-acetyltransferase can be derived from Acinetobacter baumannii, Aeromonas hydrophilia, Alcaligenes latus, Arthrospira platensis, Bacillus subtilis, Burkholderia cepacia, Clostridium acetobutylicum, Cupriavidus necator, Escherichia coli, Haloferax mediterranei, Pseudomonas aeruginosa, Pseudomonas fluorescens, Pseudomonas mandelii, Pseudomonas oleovorans, Pseudomonas putida, or Streptomyces coelicolor.

In one embodiment, the enzyme that converts acetoacetyl-CoA to 3-hydroxybutyryl-CoA is an acetoacetyl-CoA reductase (EC 1.1.1.36) or a 3-hydroxybutyryl-CoA dehydrogenase (EC 1.1.1.157). For example, the acetoacetyl-CoA reductase can be derived from Acinetobacter baumannii, Aeromonas hydrophilia, Alcaligenes latus, Arthrospira platensis, Bacillus subtilis, Burkholderia cepacia, Cupriavidus necator, Haloferax mediterranei, Pseudomonas aeruginosa, Pseudomonas fluorescens, Pseudomonas mandelii, Pseudomonas oleovorans, Pseudomonas putida, or Streptomyces coelicolor. In another example, the 3-hydroxybutyryl-CoA dehydrogenase can be derived from Clostridium beijerinckii, Clostridium acetobutylicum, or Clostridium kluyveri.

In one embodiment, the enzyme that converts 3-hydroxybutyryl-CoA to polyhydroxybutyrate is a polyhydroxyalkanoate synthase (EC 2.3.1.-). For example, the polyhydroxyalkanoate synthase can be derived from Acinetobacter baumannii, Aeromonas caviae, Aeromonas hydrophilia, Alcaligenes latus, Arthrospira platensis, Bacillus subtilis, Burkholderia cepacia, Cupriavidus necator, Haloferax mediterranei, Pseudomonas aeruginosa, Pseudomonas fluorescens, Pseudomonas mandelii, Pseudomonas oleovorans, Pseudomonas putida, Pseudomonas sp. 61-3, Rhodospirillum rubrum, or Streptomyces coelicolor.

In one embodiment, the microorganism is a member of a genus selected from the group consisting of Acetobacterium, Alkalibaculum, Blautia, Butyribacterium, Clostridium, Eubacterium, Moorella, Oxobacter, Sporomusa, and Thermoanaerobacter. For example, the microorganism can be derived from a parental microorganism selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi. In a preferred embodiment, the microorganism is derived from a parental bacterium selected from the group consisting of Clostridium autoethanogenum, Clostridium coskatii, Clostridium ljungdahlii, and Clostridium ragsdalei.

In one embodiment, the microorganism consumes gaseous substrates comprising one or more of CO, CO₂, and H₂. In another embodiment, the microorganism is anaerobic. In yet another embodiment, the microorganism is not capable of degrading PHB.

The invention further provides a method of producing PHB comprising culturing the microorganism of the invention in the presence of a gaseous substrate. For example, the gaseous substrate can comprise one or more of CO, CO₂, and H₂. In one embodiment, the culturing is performed under anaerobic conditions. In another embodiment, the culturing is performed in the absence of carbohydrate substrates. In yet another embodiment, the culturing is performed in the absence of light.

These and other features and advantages of the present invention will be more fully understood from the following detailed description taken together with the accompanying claims. It is noted that the scope of the claims is defined by the recitations therein and not by the specific discussion of features and advantages set forth in the present description.

### DESCRIPTION OF THE DRAWINGS

FIG. 1 is a diagram showing an enzymatic pathway to polyhydroxybutyrate (PHB) production.

FIG. 2 is a plasmid map of pPHB_01.

FIG. 3 is a graph showing PHB production in C. autoethanogenum carrying either a plasmid with a PHB biosynthesis pathway (pPHB_01) or an empty plasmid (pMTL83157) as a negative control. The bacteria were grown anaerobically in pressure-rated bottles with only CO and CO₂ as carbon sources. Yield of PHB (expressed as weight % of dried cell mass) was determined by HPLC. Values represent averages of biological triplicates plus or minus the standard deviations about those averages. PHB was not detected in samples comprising the pMTL83157 (empty) plasmid.

FIGS. 4A-4D are graphs showing growth of C. autoethanogenum under various growth conditions. Bacteria comprised either an empty plasmid (pMTL83157) or a plasmid comprising the PHB synthesis pathway (pPHB_01). Each plot represents a different set of conditions. FIG. 4A (condition 1) shows a repeat of conditions used to generate the data observed in FIG. 3, using a gas mix comprising 50/18/3/29 of CO/CO₂/H₂/N₂ as the sole carbon source. FIG. 4B (condition 2) shows identical conditions as in condition 1 but with a new gas substrate (50/30/10/10 CO/CO₂/H₂/N₂). FIG. 4C (condition 3) shows identical conditions as in condition 2 but with an extended incubation time. FIG. 4D (condition 4) shows identical conditions as condition 3 but with periodical refreshing of the gas substrate. Values represent averages of biological triplicates plus or minus the standard deviations about those averages.

FIG. 5 is a graph showing PHB production in C. autoethanogenum under conditions 1-4, as described in connection with FIGS. 4A-4D. Values represent averages of biological triplicates plus or minus the standard deviations about those averages. PHB was not detected in samples comprising the pMTL83157 (empty) plasmid.

FIG. 6 is a graph showing PHB production in C. autoethanogenum from gaseous carbon sources in a continuous fermentation. The bacteria were conjugated with the plasmid pPHB_01 comprising a PHB synthesis pathway. PHB was measured at completion of the fermentation. Gas substrates comprised either 20% hydrogen or 2% hydrogen as part of their mixtures of 50/20/20/10 CO/CO₂/H₂/Ar or 50/20/2/28 CO/CO₂/H₂/N₂, respectively. A pH of 5 was maintained. Values represent averages of duplicate fermentations plus or minus the standard deviations about those averages.

FIG. 7 is a graph showing enhanced PHB production in C. autoethanogenum from gaseous carbon sources in a continuous fermentation. The bacteria were conjugated with the plasmid pPHB_01 comprising a PHB synthesis pathway. PHB was measured at completion of the fermentation. Gas substrates comprised either 20% hydrogen or 2% hydrogen as part of their mixtures of 50/20/20/10 CO/CO₂/H₂/Ar or 50/20/2/28 CO/CO₂/H₂/N₂, respectively. 20% hydrogen, low biomass had a decreased concentration of biomass compared to the other runs. 20% hydrogen, varied pH started with a pH of 6 and then reduced to 5.5. 20% hydrogen, pH 6 was maintained for the entirety of the fermentation run until cultures declined. Values represent averages of duplicate fermentations plus or minus the standard deviations about those averages.

FIG. 8 is a graph showing PHB values for simulations using genome-scale metabolic model reconstructions (GEM). PHB experimentally detected was compared to the levels of PHB predicted by GEM using the maximization of PHB yield. Maximization of PHB yield was also tested with an uptake of 2 mmol/gDCW/h of either NADH, NADPH, Fd_{red}, or ATP. The conditions tested were PHB20 (control); PHBLowB (low biomass) and PHBpH5.5 (pH 5.5). ATP was found to be limiting PHB the most (highest PHB value achieved), followed by Fd_{red}, NADPH, and then NADH. Data represent an average±standard error of two biological replicates chemostats.

### DESCRIPTION OF THE INVENTION

It has long been recognized that catalytic processes, such as the Fischer-Tropsch process, may be used to convert gases comprising carbon dioxide (CO₂), carbon monoxide (CO), and/or hydrogen (H₂), such as industrial waste gas or syngas, into a variety of fuels and chemicals. Recently, however, gas fermentation has emerged as an alternative platform for the biological fixation of such gases. In particular, acetogenic (i.e., Wood-Ljungdahl) microorganisms have been demonstrated to convert gases comprising CO, CO₂, and/or H₂ into products such as ethanol and 2,3-butanediol. The desirability of producing more complex polymer molecules, such as PHB, from these gases is well-documented (Drzyzga, J Chem Technol Biotechnol, 90: 1735-1751, 2015). However, the Wood-Ljungdahl pathway operates at the thermodynamic edge of life (Schuchmann, Nat Rev Microbiol, 12: 809-821, 2014), which makes it difficult for Wood-Ljungdahl microorganisms to accumulate even enough carbon for cell growth and maintenance, much less produce complex carbon products. These metabolic challenges are compounded by poor dissolution of gaseous substrates (e.g., CO, CO₂, and/or H₂) in fermentation media compared to carbohydrate or sugar substrates. Therefore, it would appear unlikely that Wood-Ljungdahl microorganisms could be engineered to synthesize PHB or other polyhydroxyalkanoates, especially since these polymers are natively produced by species such as Rhodospirillum rubrum and Cupriavidus necator as a means to store excess carbon. Indeed, to date, attempts to engineer acetogenic microorganisms to produce PHB from CO, CO₂, and/or H₂ have been unsuccessful (The European SYNPOL Project, Biopolymers from syngas fermentation, 2012-2017).

After diligent research and engineering efforts, however, the inventors have achieved the first-ever synthesis of PHB in Wood-Ljungdahl microorganisms. This represents a major milestone on the path to the production renewable and sustainable biopolymers.

In a first aspect, the invention provides a Wood-Ljungdahl microorganism capable of producing PHB. In a second aspect, the invention provides a method of producing PHB by culturing the aforementioned Wood-Ljungdahl microorganism in the presence of a gaseous substrate.

### Pathway

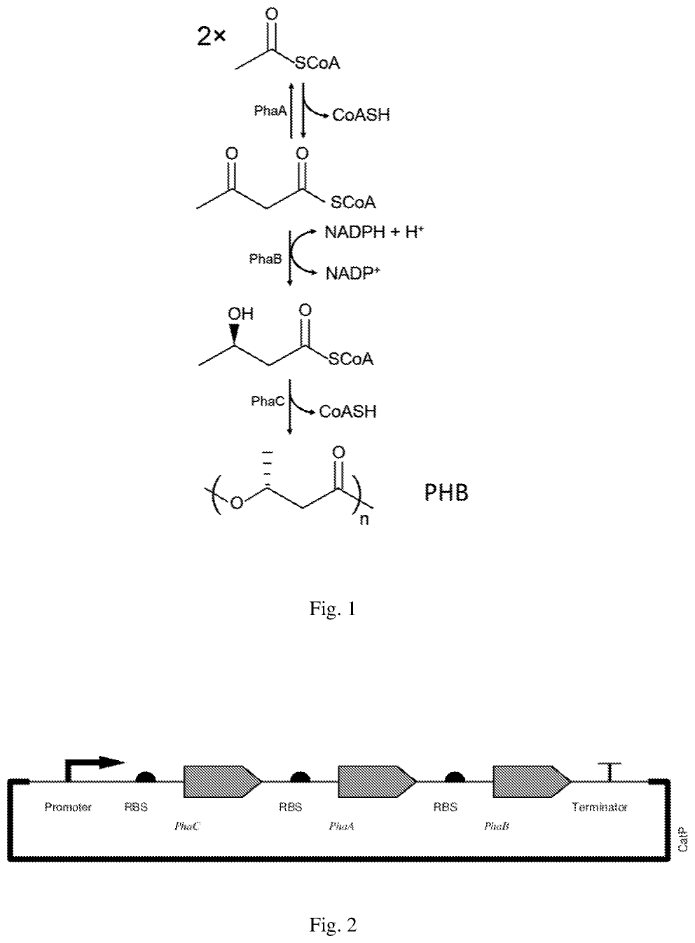

Since Wood-Ljungdahl microorganisms do not natively produce PHB, the production of PHB in a Wood-Ljungdahl microorganism requires the introduction of at least one heterologous enzyme. The microorganism of the invention generally comprises three heterologous enzymes, namely (a) an enzyme that converts acetyl-CoA to acetoacetyl-CoA, (b) an enzyme that converts acetoacetyl-CoA to 3-hydroxybutyryl-CoA, and (c) an enzyme that converts 3-hydroxybutyryl-CoA to polyhydroxybutyrate. This pathway is depicted in FIG. 1.

### (1) Conversion of Acetyl-CoA to Acetoacetyl-CoA

The conversion of acetyl-CoA to acetoacetyl-CoA may be catalyzed by any suitable enzyme. Although it is possible that native activity for this reaction may be present in certain acetogenic bacteria, it is usually necessary to introduce a heterologous (i.e., non-native) enzyme to catalyze this reaction. In a preferred embodiment, the enzyme is acetyl-CoA C-acetyltransferase (also known as thiolase or 3-ketothiolase), which has activity defined by EC 2.3.1.9 (i.e., 2 acetyl-CoA 4↔CoA+acetoacetyl-CoA). The acetyl-CoA C-acetyltransferase may be derived from any suitable host microorganism, such as Acinetobacter baumannii, Aeromonas hydrophilia, Alcaligenes latus, Arthrospira platensis, Bacillus subtilis, Burkholderia cepacia, Clostridium acetobutylicum, Cupriavidus necator, Escherichia coli, Haloferax mediterranei, Pseudomonas aeruginosa, Pseudomonas fluorescens, Pseudomonas mandelii, Pseudomonas oleovorans, Pseudomonas putida, or Streptomyces coelicolor.

In particular, the acetyl-CoA C-acetyltransferase may be or may be derived from Acinetobacter baumannii PhaA (SCZ16966), Aeromonas hydrophilia PhaA (WP_043162470), Alcaligenes latus PhaA (AAC83659), Arthrospira platensis PhaA (WP_006617472), Bacillus subtilis PhaA (CUB52080), Burkholderia cepacia PhaA (WP_043187452), Clostridium acetobutylicum ThlA (WP_0109661571), Cupriavidus necator PhaA (WP_013956452.1), Cupriavidus necator BktB (WP_011615089.1), Cupriavidus necator phaA (WP_010810132.1), Escherichia coli AtoB (NP_416728.1), Haloferax mediterranei PhaA (WP_004059344), Pseudomonas aeruginosa PhaA (WP_038823536), Pseudomonas fluorescens PhaA (WP_073525707), Pseudomonas mandelii PhaA (WP_019582144), Pseudomonas oleovorans PhaA (WP_074859314), Pseudomonas putida PhaA (WP_058540218), or Streptomyces coelicolor PhaA (WP_011030221).

### (2) Conversion of Acetoacetyl-CoA to 3-Hydroxybutyryl-CoA

The conversion of acetoacetyl-CoA to 3-hydroxybutyryl-CoA may be catalyzed by any suitable enzyme. Although it is possible that native activity for this reaction may be present in certain acetogenic bacteria, it is usually necessary to introduce a heterologous (i.e., non-native) enzyme to catalyze this reaction. In a preferred embodiment, the enzyme is acetoacetyl-CoA reductase, which has activity defined by EC 1.1.1.36 (i.e., (R)-3-hydroxyacyl-CoA+NADP⁺↔3-oxoacyl-CoA+NADPH+H⁺). The acetoacetyl-CoA reductase may be derived from any suitable host microorganism, such as Acinetobacter baumannii, Aeromonas hydrophilia, Alcaligenes latus, Arthrospira platensis, Bacillus subtilis, Burkholderia cepacia, Cupriavidus necator, Haloferax mediterranei, Pseudomonas aeruginosa, Pseudomonas fluorescens, Pseudomonas mandelii, Pseudomonas oleovorans, Pseudomonas putida, or Streptomyces coelicolor. In particular, the acetoacetyl-CoA reductase may be Acinetobacter baumannii PhaB (WP_095389464), Aeromonas hydrophilia PhaB (WP_041216919), Alcaligenes latus PhaB (AAC83660), Arthrospira platensis PhaB (WP_043469113), Bacillus subtilis PhaB (WP_070548955), Burkholderia cepacia PhaB (WP_059234032), Cupriavidus necator PhaB (WP_010810131.1), Haloferax mediterranei PhaB (WP_004572392), Pseudomonas aeruginosa PhaB (WP_031690879), Pseudomonas fluorescens PhaB (WP_030141425), Pseudomonas mandelii PhaB (WP_094467462), Pseudomonas oleovorans PhaB (WP_074858624), Pseudomonas putida PhaB (BAB96554), or Streptomyces coelicolor PhaB (WP_011027734). In another preferred embodiment, the enzyme is 3-hydroxybutyryl-CoA dehydrogenase, which has activity defined by EC 1.1.1.157 (i.e., (S)-3-hydroxybutanoyl-CoA+NADP⁺=3-acetoacetyl-CoA+NADPH+H⁺). The 3-hydroxybutyryl-CoA dehydrogenase may be or may be derived from derived from any suitable host microorganism, such as Clostridium beijerinckii, Clostridium acetobutylicum, or Clostridium kluyveri. In particular, the 3-hydroxybutyryl-CoA dehydrogenase may be Clostridium beijerinckii Hbd (WP_011967675.1), Clostridium acetobutylicum Hbd (NP_349314.1), or Clostridium kluyveri Hbd1 (WP_011989027.1).

The conversion of acetoacetyl-CoA to 3-hydroxybutyryl-CoA may be catalyzed by 3-hydroxybutyryl-CoA dehydrogenase (EC 1.1.1.157). The 3-hydroxybutyryl-CoA dehydrogenase may be, for example, Hbd from Clostridium beijerinckii (WP_011967675.1), Hbd from Clostridium acetobutylicum (NP_349314.1), or Hbd1 from Clostridium kluyveri (WP_011989027.1). Moreover, this step may also be catalyzed by acetoacetyl-CoA reductase (EC 4.2.1.36). The acetoacetyl-CoA reductase may be, for example, PhaB from Cupriavidus necator (WP_010810131.1) (SEQ ID NO: 58). This step may also be catalyzed by acetoacetyl-CoA hydratase (EC 4.2.1.119). Of note, PhaB is R-specific and Hbd is 5-specific. Additionally, Hbd1 from Clostridium kluyveri is NADPH-dependent and Hbd from Clostridium acetobutylicum and Clostridium beijerinckii are NADH-dependent. Clostridium autoethanogenum, Clostridium ljungdahlii, and Clostridium ragsdalei do not have known native activity for this step. Escherichia coli does not have known native activity for this step.

Preferably, the enzyme that converts acetoacetyl-CoA to 3-hydroxybutyryl-CoA is (R)-specific, i.e., produces (R)-3-hydroxybutyryl-CoA, since (R)-3-hydroxybutyryl-CoA is the typical substrate for the enzymatic production of PHB. However, in some instances, the enzyme that converts acetoacetyl-CoA to 3-hydroxybutyryl-CoA is (S)-specific, i.e., produces (S)-3-hydroxybutyryl-CoA. Without wishing to be bound by any particular theory, the inventors believe that native or introduced epimerase activity in acetogenic bacteria may allow for the interconversion of (S)- and (R)-3-hydroxybutyryl-CoA, such (S)-3-hydroxybutyryl-CoA may be converted to (R)-3-hydroxybutyryl-CoA, which may then be converted to PHB.

### (3) Conversion of 3-Hydroxybutyryl-CoA to PHB

The conversion of 3-hydroxybutyryl-CoA to PHB may be catalyzed by any suitable enzyme. Although it is possible that native activity for this reaction may be present in certain acetogenic bacteria, it is usually necessary to introduce a heterologous (i.e., non-native) enzyme to catalyze this reaction. In a preferred embodiment, the enzyme is polyhydroxyalkanoate synthase, which has activity defined by EC 2.3.1.-, such as EC 2.3.1.B2 (type I) (i.e., 3-hydroxybutyryl-CoA+[(R)-3-hydroxybutanoate]ₐ=[(R)-3-hydroxybutanoate]ₙ₊₁+CoA), EC 2.3.1.B3 (type II) (i.e., 3-hydroxyacyl-CoA+[(R)-3-hydroxyacyl]ₙ=[(R)-3-hydroxyacyl]ₙ₊₁+CoA), or EC 2.3.1.B4 (type III) (i.e., 3-hydroxyacyl-CoA+[(R)-3-hydroxyacyl]ₙ=[(R)-3-hydroxyacyl]ₙ₊₁+CoA). This enzyme may also be referred to as polyhydroxyalkanoate polymerase, polyhydroxybutyrate synthase, polyhydroxybutyrate polymerase, and the like. The polyhydroxyalkanoate synthase may be derived from any suitable host microorganism, such as Acinetobacter baumannii, Aeromonas caviae, Aeromonas hydrophilia, Alcaligenes latus, Arthrospira platensis, Bacillus subtilis, Burkholderia cepacia, Cupriavidus necator, Haloferax mediterranei, Pseudomonas aeruginosa, Pseudomonas fluorescens, Pseudomonas mandelii, Pseudomonas oleovorans, Pseudomonas putida, Pseudomonas sp. 61-3, Rhodospirillum rubrum, or Streptomyces coelicolor. In particular, the polyhydroxyalkanoate synthase may be or may be derived from Acinetobacter baumannii PhaC (SCY71072), Aeromonas caviae PhaC (WP_045524574), Aeromonas hydrophilia PhaC1 (WP_017780191) or PhaC2 (AAV41872), Alcaligenes latus PhaC (WP_084267317), Arthrospira platensis PhaC (WP_006617456), Bacillus subtilis PhaC (CUB58881), Burkholderia cepacia PhaC (WP_027784567), Cupriavidus necator PhaC (WP_011615085 or WP_013956451.1), Haloferax mediterranei PhaC (WP_004056138), Pseudomonas aeruginosa PhaC1 (WP_038823539) or PhaC2 (WP_025271419), Pseudomonas fluorescens PhaC1 (WP_057399292) or PhaC2 (WP_030141001), Pseudomonas mandelii PhaC1 (WP_094467460) or PhaC2 (WP_010465951), Pseudomonas oleovorans PhaC1 (AAL17611) or PhaC2 (WP_037049875), Pseudomonas putida PhaC1 (BAB96552) or PhaC2 (WP_029886362), Pseudomonas sp. 61-3 PhaC1 (BAA36198) or PhaC2 (BAA36202), Rhodospirillum rubrum PhaC1 (WP_011388028), PhaC2 (WP_011390166), or PhaC3 (WP_011398569), or Streptomyces coelicolor PhaC.

One embodiment provides a recombinant carboxydotrophic Clostridia microorganism adapted to express one or more exogenous NADPH-dependent enzymes, and/or adapted to over-express one or more endogenous NADPH-dependent enzymes, the enzymes selected such that when the exogenous enzyme is expressed, and/or the endogenous enzyme is overexpressed, the overall utilisation of NADPH by the microorganism is increased relative to a parental microorganism.

In one particular embodiment, the increase in overall utilisation of NADPH results, in use, in an increase in the efficiency of production of one or more products by the microorganism.

In a particular embodiment, the microorganism is adapted to express and/or over-express an NADPH-dependent isoform while the expression of a corresponding NADH-dependent isoform is substantially unchanged, decreases, or exhibits a comparatively smaller increase when compared to the change in expression of the NADPH-dependent isoform. In one particular embodiment, the microorganism is adapted so expression of the one or more NADH-dependent isoforms is attenuated or knocked out compared to a parental microorganism. In one embodiment, the expression is attenuated or knocked out by modifying a nucleic acid encoding the one or more NADH-dependent enzyme or replacing one or more nucleic acid encoding an NADH-dependent isoform with one or more nucleic acid encoding an NADPH-dependent isoform.

In one embodiment, the one or more enzymes existing in NADPH- and NADH-dependent isoforms is a hydroxybutyryl-CoA dehydrogenase/acetoacetyl-CoA reductase/3-hydroxybutyryl-CoA hydratase, and comprises an NADPH-dependent isoform phaB (EC:1.1.1.36; GO:0018454; e.g. from Ralstonia eutropha: YP_725942.1, GeneID:4249784 or a functionally equivalent variant of any one thereof), NADPH dependent phaJ (EC 4.2.1.119; e.g. from Aeromonas punctata: BAA21816.1) and a corresponding NADH-dependent isoform hbd (EC 1.1.1.157; GO:0008691; e.g. from C. acetobutylicum: NP_349314.1, GeneID:1118891 or a functionally equivalent variant of any one thereof).

In another embodiment, the recombinant microorganism exhibits attenuated expression of one or more NADH-dependent enzymes. In this embodiment, an NADH-dependent isoform of an enzyme in a parental microorganism may have been replaced by an NADPH-dependent isoform of the enzyme in the recombinant microorganism.

In a particular embodiment, the microorganism exhibits increased efficiency during a fermentation reaction when compared to a parental microorganism.

In a further embodiment, the one or more NADPH-dependent enzymes is modified to increase its NADPH co-factor specificity relative to its NADH co-factor specificity.

In a further embodiment, the method further comprises a step of increasing the NADPH co-factor specificity of the one or more NADPH-dependent enzymes relative to the NADH co-factor specificity of the enzyme(s). In one embodiment, this comprises modifying one or more nucleic acid encoding one or more NADPH-dependent enzymes.

In certain embodiments, one or more disruptive mutations may be introduced to one or more endogenous enzymes to reduce or eliminate competition with introduced heterologous enzymes. In particular, a “disruptive mutation” is a mutation that reduces or eliminates (i.e., “disrupts”) the expression or activity of a gene or enzyme. The disruptive mutation may partially inactivate, fully inactivate, or delete the gene or enzyme. The disruptive mutation may be a knockout (KO) mutation. The disruptive mutation may be any mutation that reduces, prevents, or blocks the biosynthesis of a product produced by an enzyme. The disruptive mutation may include, for example, a mutation in a gene encoding an enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, siRNA, guide RNA) and/or protein (e.g., a Cas protein) which inhibits the expression of an enzyme. The disruptive mutation may be introduced using any method known in the art.

For example, the microorganism of the invention may have a disruptive mutation in an endogenous thioesterase enzyme. Three putative thioesterases have been identified in Clostridium autoethanogenum: (1) “thioesterase 1” (AGY74947.1; annotated as palmitoyl-CoA hydrolase), (2) “thioesterase 2” (AGY75747.1; annotated as 4-hydroxybenzoyl-CoA thioesterase), and (3) “thioesterase 3” (AGY75999.1; annotated as putative thioesterase). Three putative thioesterases have also been identified in Clostridium ljungdahlii: (1) “thioesterase 1” (ADK15695.1; annotated as predicted acyl-CoA thioesterase 1), (2) “thioesterase 2” (ADK16655.1; annotated as predicted thioesterase), and (3) “thioesterase 3” (ADK16959.1; annotated as predicted thioesterase). The disruptive mutation may affect any of these thioesterases or any other thioesterases that may be endogenous to the microorganism of the invention.

### Microorganism

A “microorganism” is a microscopic organism, especially a bacterium, archea, virus, or fungus. The microorganism of the invention is typically a bacterium. As used herein, recitation of “microorganism” should be taken to encompass “bacterium.”

The microorganism of the invention is non-naturally occurring. The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility. In contrast, “wild-type” refers to the typical form of an organism, strain, gene, or characteristic as it occurs in nature.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refer to a microorganism comprising such a genetic modification, genetic alteration, or genetic engineering. These terms may be used to differentiate a laboratory-generated microorganism from a naturally-occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization.

“Recombinant” indicates that a nucleic acid, protein, or microorganism is the product of genetic modification, engineering, or recombination. Generally, the term “recombinant” refers to a nucleic acid, protein, or microorganism that comprises or is encoded by genetic material derived from multiple sources, such as two or more different strains or species of microorganisms. The microorganism of the invention is typically recombinant.

The term “derived from” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes. Generally, the microorganism of the invention is derived from a “parental microorganism,” which is a microorganism used to generate a microorganism of the invention. The parental microorganism may be a naturally-occurring microorganism (i.e., a wild-type microorganism) or a microorganism that has been previously modified (i.e., a mutant or recombinant microorganism). The microorganism of the invention may be modified to express or overexpress one or more enzymes that were not expressed or overexpressed in the parental microorganism. Similarly, the microorganism of the invention may be modified to comprise one or more genes that were not comprised by the parental microorganism. The microorganism of the invention may also be modified to not express or to express lower amounts of one or more enzymes that were expressed in the parental microorganism. In one embodiment, the microorganism of the invention is derived from a parental microorganism selected from the group consisting of Clostridium autoethanogenum, Clostridium ljungdahlii, or Clostridium ragsdalei. In a preferred embodiment, the microorganism of the invention is derived from the parental microorganism Clostridium autoethanogenum LZ1561, which was deposited on Jun. 7, 2010 with Deutsche Sammlung von Mikroorganismen and Zellkulturen GmbH (DSMZ) located at Inhoffenstraße 7B, D-38124 Braunschwieg, Germany on Jun. 7, 2010 under the terms of the Budapest Treaty and accorded accession number DSM23693. This strain is described in International Patent Application No. PCT/NZ2011/000144, which published as WO 2012/015317.

The microorganism of the invention may be further classified based on functional characteristics. For example, the microorganism of the invention may be or may be derived from a Wood-Ljungdahl microorganism, a C1-fixing microorganism, an anaerobe, an acetogen, an ethanologen, and/or a carboxydotroph. Table 1 provides a representative list of microorganisms and identifies their functional characteristics.

TABLE 1

| | Wood-Ljungdahl | C1-fixing | Anaerobe | Acetogen | Ethanologen | Autotroph | Carboxydotroph |
|---|---|---|---|---|---|---|---|
| Acetobacterium woodii | + | + | + | + | +/− ¹ | + | − |
| Alkalibaculum bacchii | + | + | + | + | + | + | + |
| Blautia producta | + | + | + | + | − | + | + |
| Butyribacterium methylotrophicum | + | + | + | + | + | + | + |
| Clostridium aceticum | + | + | + | + | − | + | + |
| Clostridium autoethanogenum | + | + | + | + | + | + | + |
| Clostridium carboxidivorans | + | + | + | + | + | + | + |
| Clostridium coskatii | + | + | + | + | + | + | + |
| Clostridium drakei | + | + | + | + | − | + | + |
| Clostridium formicoaceticum | + | + | + | + | − | + | + |
| Clostridium ljungdahlii | + | + | + | + | + | + | + |
| Clostridium magnum | + | + | + | + | − | + | +/− ² |
| Clostridium ragsdalei | + | + | + | + | + | + | + |
| Clostridium scatologenes | + | + | + | + | − | + | + |
| Eubacterium limosum | + | + | + | + | − | + | + |
| Moorella thermautotrophica | + | + | + | + | + | + | + |
| Moorella thermoacetica (formerly | + | + | + | + | − ³ | + | + |
| Clostridium thermoaceticum) | | | | | | | |
| Oxobacter pfennigii | + | + | + | + | − | + | + |
| Sporomusa ovata | + | + | + | + | − | + | +/− ⁴ |
| Sporomusa silvacetica | + | + | + | + | − | + | +/− ⁵ |
| Sporomusa sphaeroides | + | + | + | + | − | + | +/− ⁶ |
| Thermoanaerobacter kivui | + | + | + | + | − | + | − |

¹ Acetobacterium woodii can produce ethanol from fructose, but not from gas.
² It has not been investigated whether Clostridium magnum can grow on CO.
³ One strain of Moorella thermoacetica, Moorella sp. HUC22-1, has been reported to produce ethanol from gas.
⁴ It has not been investigated whether Sporomusa ovata can grow on CO.
⁵ It has not been investigated whether Sporomusa silvacetica can grow on CO.
⁶ It has not been investigated whether Sporomusa sphaeroides can grow on CO.

“Wood-Ljungdahl” refers to the Wood-Ljungdahl pathway of carbon fixation as described, e.g., by Ragsdale, Biochim Biophys Acta, 1784: 1873-1898, 2008. “Wood-Ljungdahl microorganisms” refers, predictably, to microorganisms comprising the Wood-Ljungdahl pathway. Generally, the microorganism of the invention comprises a native Wood-Ljungdahl pathway. Herein, a Wood-Ljungdahl pathway may be a native, unmodified Wood-Ljungdahl pathway or it may be a Wood-Ljungdahl pathway with some degree of genetic modification (e.g., overexpression, heterologous expression, knockout, etc.) so long as it still functions to convert CO, CO₂, and/or H₂ to acetyl-CoA.

“C1” refers to a one-carbon molecule, for example, CO, CO₂, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the invention. For example, a C1-carbon source may comprise one or more of CO, CO₂, CH₃OH, or CH₂O₂. Preferably, the C1-carbon source comprises one or both of CO and CO₂. A “C1-fixing microorganism” is a microorganism that has the ability to produce one or more products from a C1-carbon source. Typically, the microorganism of the invention is a C1-fixing bacterium. In a preferred embodiment, the microorganism of the invention is derived from a C1-fixing microorganism identified in Table 1. For the purposes of the present invention, methane (CH₄) could be considered a C1-carbon source, but only if the bacterium of the invention was engineered to comprise a methane metabolic pathway, as described, e.g., in WO 2016/138050, since acetogenic bacteria are not natively capable of using methane as a carbon source.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen). Typically, the microorganism of the invention is an anaerobe. In a preferred embodiment, the microorganism of the invention is derived from an anaerobe identified in Table 1.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). Acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, N.Y., 2006). All naturally occurring acetogens are C1-fixing, anaerobic, autotrophic, and non-methanotrophic. Typically, the microorganism of the invention is an acetogen. In a preferred embodiment, the microorganism of the invention is derived from an acetogen identified in Table 1.

An “ethanologen” is a microorganism that produces or is capable of producing ethanol. Typically, the microorganism of the invention is an ethanologen. In a preferred embodiment, the microorganism of the invention is derived from an ethanologen identified in Table 1.

An “autotroph” is a microorganism capable of growing in the absence of organic carbon. Instead, autotrophs use inorganic carbon sources, such as CO and/or CO₂. Typically, the microorganism of the invention is an autotroph. In a preferred embodiment, the microorganism of the invention is derived from an autotroph identified in Table 1.

A “carboxydotroph” is a microorganism capable of utilizing CO as a sole source of carbon and energy. Typically, the microorganism of the invention is a carboxydotroph. In a preferred embodiment, the microorganism of the invention is derived from a carboxydotroph identified in Table 1.

More broadly, the microorganism of the invention may be derived from any genus or species identified in Table 1. For example, the microorganism may be a member of a genus selected from the group consisting of Acetobacterium, Alkalibaculum, Blautia, Butyribacterium, Clostridium, Eubacterium, Moorella, Oxobacter, Sporomusa, and Thermoanaerobacter. In particular, the microorganism may be derived from a parental bacterium selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi.

In a preferred embodiment, the microorganism of the invention is derived from the cluster of Clostridia comprising the species Clostridium autoethanogenum, Clostridium coskatii, Clostridium ljungdahlii, and Clostridium ragsdalei. These species were first reported and characterized by Abrini, Arch Microbiol, 161: 345-351, 1994 (Clostridium autoethanogenum), Tanner, Int J System Bacteriol, 43: 232-236, 1993 (Clostridium ljungdahlii), and Huhnke, WO 2008/028055 (Clostridium ragsdalei).

These species have many similarities. In particular, these species are all C1-fixing, anaerobic, acetogenic, ethanologenic, and carboxydotrophic members of the genus Clostridium. These species have similar genotypes and phenotypes and modes of energy conservation and fermentative metabolism. Moreover, these species are clustered in clostridial rRNA homology group I with 16S rRNA DNA that is more than 99% identical, have a DNA G+C content of about 22-30 mol %, are gram-positive, have similar morphology and size (logarithmic growing cells between 0.5-0.7×3-5 μm), are mesophilic (grow optimally at 30-37° C.), have similar pH ranges of about 4-7.5 (with an optimal pH of about 5.5-6), lack cytochromes, and conserve energy via an Rnf complex. Also, reduction of carboxylic acids into their corresponding alcohols has been shown in these species (Perez, Biotechnol Bioeng, 110:1066-1077, 2012). Importantly, these species also all show strong autotrophic growth on CO-comprising gases, produce ethanol and acetate (or acetic acid) as main fermentation products, and produce small amounts of 2,3-butanediol and lactic acid under certain conditions.

However, these species also have a number of differences. These species were isolated from different sources: Clostridium autoethanogenum from rabbit gut, Clostridium ljungdahlii from chicken yard waste, and Clostridium ragsdalei from freshwater sediment. These species differ in utilization of various sugars (e.g., rhamnose, arabinose), acids (e.g., gluconate, citrate), amino acids (e.g., arginine, histidine), and other substrates (e.g., betaine, butanol). Moreover, these species differ in auxotrophy to certain vitamins (e.g., thiamine, biotin). These species have differences in nucleic and amino acid sequences of Wood-Ljungdahl pathway genes and proteins, although the general organization and number of these genes and proteins has been found to be the same in all species (Köpke, Curr Opin Biotechnol, 22: 320-325, 2011).

Thus, in summary, many of the characteristics of Clostridium autoethanogenum, Clostridium coskatii, Clostridium ljungdahlii, or Clostridium ragsdalei are not specific to that species, but are rather general characteristics for this cluster of C1-fixing, anaerobic, acetogenic, ethanologenic, and carboxydotrophic members of the genus Clostridium. However, since these species are, in fact, distinct, the genetic modification or manipulation of one of these species may not have an identical effect in another of these species. For instance, differences in growth, performance, or product production may be observed.

The microorganism of the invention may also be derived from an isolate or mutant of Clostridium autoethanogenum, Clostridium coskatii, Clostridium ljungdahlii, or Clostridium ragsdalei. Isolates and mutants of Clostridium autoethanogenum include JA1-1 (DSM10061) (Abrini, Arch Microbiol, 161: 345-351, 1994), LBS1560 (DSM19630) (WO 2009/064200), and LZ1561 (DSM23693) (WO 2012/015317). Isolates and mutants of Clostridium ljungdahlii include ATCC 49587 (Tanner, Int J Syst Bacteriol, 43: 232-236, 1993), PETCT (DSM13528, ATCC 55383), ERI-2 (ATCC 55380) (U.S. Pat. No. 5,593,886), C-01 (ATCC 55988) (U.S. Pat. No. 6,368,819), 0-52 (ATCC 55989) (U.S. Pat. No. 6,368,819), and OTA-1 (Tirado-Acevedo, Production of bioethanol from synthesis gas using Clostridium ljungdahlii, PhD thesis, North Carolina State University, 2010). Isolates and mutants of Clostridium ragsdalei include PI 1 (ATCC BAA-622, ATCC PTA-7826) (WO 2008/028055).

Preferably, the microorganism of the invention is not phototrophic or photosynthetic. Preferably, the microorganism of the invention is not methanotrophic.

Preferably, the microorganism of the invention is not a member of the genus Alcaligenes, Azotobacter, Bacillus, Cupriavidus (Ralstonia), Rhizobium, Rhodospirillum, or Pseudomonas. In particular, the microorganism of the invention is preferably not derived from Rhodospirillum rubrum, Bacillus cereus, Cupriavidus necator (formerly Ralstonia eutropha), or Pseudomonas putida. In other embodiments, the microorganism of the invention is preferably not derived from Escherichia coli.

### Enzymes

“Endogenous” or “native” refers to a nucleic acid or protein that is present or expressed in the wild-type or parental microorganism from which the microorganism of the invention is derived. For example, an endogenous gene or protein is a gene or protein that is natively present in the wild-type or parental microorganism from which the microorganism of the invention is derived. In one embodiment, the expression of an endogenous gene may be controlled by an exogenous regulatory element, such as an exogenous promoter.

“Exogenous” refers to a nucleic acid or protein that originates outside the microorganism of the invention. For example, an exogenous gene or enzyme may be artificially or recombinantly created and introduced to or expressed in the microorganism of the invention. An exogenous gene or enzyme may also be isolated from a heterologous microorganism and introduced to or expressed in the microorganism of the invention. Exogenous nucleic acids may be adapted to integrate into the genome of the microorganism of the invention or to remain in an extra-chromosomal state in the microorganism of the invention, for example, in a plasmid.

“Heterologous” refers to a nucleic acid or protein that is not present in the wild-type or parental microorganism from which the microorganism of the invention is derived. For example, a heterologous gene or enzyme may be derived from a different strain or species and introduced to or expressed in the microorganism of the invention.

Typically, at least one of the enzymes that (a) converts acetyl-CoA to acetoacetyl-CoA, (b) converts acetoacetyl-CoA to 3-hydroxybutyryl-CoA, or (c) converts 3-hydroxybutyryl-CoA to PHB is heterologous (i.e., non-native) to the bacterium. For example, one, two, or all three of these enzymes may be heterologous (i.e., non-native) to the bacterium. If the bacterium happens to have native enzymatic activity for one or more of these steps, however, it may not be necessary to introduce heterologous enzymes to catalyze those steps.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins.

“Enzyme activity,” or simply “activity,” refers broadly to enzymatic activity, including, but not limited, to the activity of an enzyme, the amount of an enzyme, or the availability of an enzyme to catalyze a reaction. Accordingly, “increasing” enzyme activity includes increasing the activity of an enzyme, increasing the amount of an enzyme, or increasing the availability of an enzyme to catalyze a reaction. Similarly, “decreasing” enzyme activity includes decreasing the activity of an enzyme, decreasing the amount of an enzyme, or decreasing the availability of an enzyme to catalyze a reaction.

“Codon optimization” refers to the mutation of a nucleic acid, such as a gene, for optimized or improved translation of the nucleic acid in a particular strain or species. Codon optimization may result in faster translation rates or higher translation accuracy. In a preferred embodiment, the genes of the invention are codon optimized for expression in Clostridium, particularly Clostridium autoethanogenum, Clostridium coskatii, Clostridium ljungdahlii, or Clostridium ragsdalei. As used herein, the terms “codon-optimized” and “codon-adapted” can be used interchangeably.

The term “variants” includes nucleic acids and proteins whose sequence varies from the sequence of a reference nucleic acid and protein, such as a sequence of a reference nucleic acid and protein disclosed in the prior art or exemplified herein. The invention may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like. Homologous genes from other microorganisms are also examples of functionally equivalent variants. These may include homologous genes in species such as Clostridium acetobutylicum, Clostridium beijerinckii, or Clostridium ljungdahlii, the details of which are publicly available on websites such as GenBank or NCBI. Functionally equivalent variants also include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

The enzymes described herein are typically expressed from a nucleic acid that has been introduced into the microorganism of the invention. Nucleic acids may be delivered to a microorganism of the invention using any method known in the art. For example, nucleic acids may be delivered as naked nucleic acids or may be formulated with one or more agents, such as liposomes. The nucleic acids may be DNA, RNA, cDNA, or combinations thereof, as is appropriate. Restriction inhibitors may be used in certain embodiments. Additional vectors may include plasmids, viruses, bacteriophages, cosmids, and artificial chromosomes. In a preferred embodiment, nucleic acids are delivered to the microorganism of the invention using a plasmid. By way of example, transformation (including transduction or transfection) may be achieved by electroporation, ultrasonication, polyethylene glycol-mediated transformation, chemical or natural competence, protoplast transformation, prophage induction, or conjugation. In certain embodiments having active restriction enzyme systems, it may be necessary to methylate a nucleic acid before introduction of the nucleic acid into a microorganism.

Furthermore, nucleic acids may be designed to comprise a regulatory element, such as a promoter, to increase or otherwise control expression of a particular nucleic acid. The promoter may be a constitutive promoter or an inducible promoter. Ideally, the promoter is a Wood-Ljungdahl pathway promoter, a ferredoxin promoter, a pyruvate:ferredoxin oxidoreductase promoter, an Rnf complex operon promoter, an ATP synthase operon promoter, or a phosphotransacetylase/acetate kinase operon promoter.

### Substrates

“Substrate” refers to a carbon and/or energy source for the microorganism of the invention. Typically, the substrate is gaseous and comprises a C1-carbon source, for example, CO and/or CO₂. Preferably, the substrate comprises a C1-carbon source of CO or CO+CO₂. The substrate may further comprise other non-carbon components, such as H₂, N₂, or electrons.

The substrate generally comprises at least some amount of CO, such as about 1, 2, 5, 10, 20, 30, 40, 50, 60, 70, 80, 90, or 100 mol % CO. The substrate may comprise a range of CO, such as about 20-80, 30-70, or 40-60 mol % CO. Preferably, the substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the substrate may comprise a relatively low amount of CO, such as about 1-10 or 1-20 mol % CO. The microorganism of the invention typically converts at least a portion of the CO in the substrate to a product. In some embodiments, the substrate comprises no or substantially no (<1 mol %) CO.

The substrate may comprise some amount of H₂. For example, the substrate may comprise about 1, 2, 5, 10, 15, 20, or 30 mol % H₂. In some embodiments, the substrate may comprise a relatively high amount of H₂, such as about 60, 70, 80, or 90 mol % H₂. In further embodiments, the substrate comprises no or substantially no (<1 mol %) H₂.

The substrate may comprise some amount of CO₂. For example, the substrate may comprise about 1-80 or 1-30 mol % CO₂. In some embodiments, the substrate may comprise less than about 20, 15, 10, or 5 mol % CO₂. In another embodiment, the substrate comprises no or substantially no (<1 mol %) CO₂.

In certain embodiments, growth of a PHB-producing strain is compared to a control (“empty plasmid” or “EP”) strain using two different CO and CO₂ containing gas mixes with either 20% of H₂ resembling syngas (50% CO, 20% CO₂, 20% H₂, 10% Argon), termed as conditions “PHB20” and “EP20,” respectively, or 2% of H₂ resembling steel mill off gas (50% CO, 20% CO₂, 2% H₂, 28% Nitrogen), termed as conditions “PHB2” and “EP2,” respectively.

Although the substrate is typically gaseous, the substrate may also be provided in alternative forms. For example, the substrate may be dissolved in a liquid saturated with a CO-comprising gas using a microbubble dispersion generator. By way of further example, the substrate may be adsorbed onto a solid support.

The substrate and/or C1-carbon source may be a waste gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the substrate and/or C1-carbon source may be captured from the industrial process before it is emitted into the atmosphere, using any convenient method.

The substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The composition of the substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

In certain embodiments, the fermentation or culturing is performed in the absence of carbohydrate substrates, such as sugar, starch, lignin, cellulose, or hemicellulose.

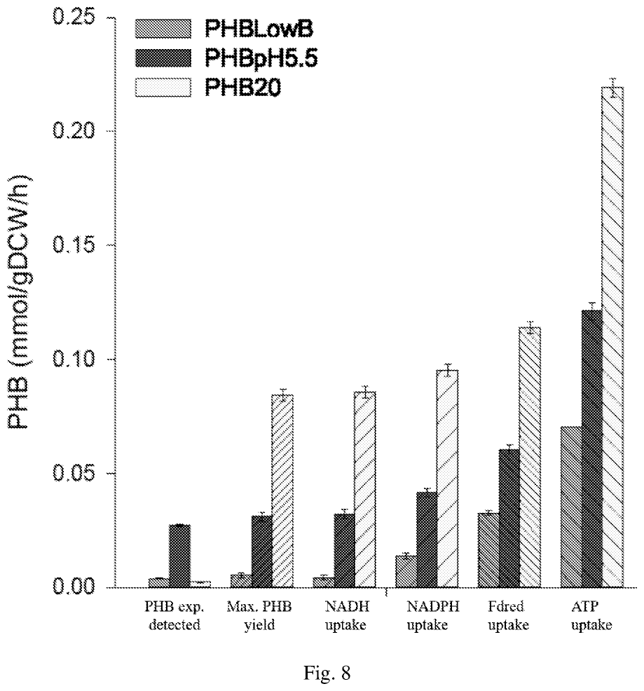

As used herein, the term “PHBLowB” is used to refer to experiments with a lower steady-state biomass concentration, such as a 3-times lower steady-state biomass concentration. As used herein, the term “PHBpH5.5” is used to refer to experiments conducted at a pH of 5.5. See FIG. 8.

### Products

The microorganism of the invention may be cultured to produce one or more products. In particular, the microorganism of the invention may produce PHB or precursors thereof, such as acetoacetyl-CoA or 3-hydroxybutyryl-CoA.

PHB is a polymer of 3-hydroxybutyrate monomers. The PHB produced according to the invention may comprise any number of 3-hydroxybutyrate monomers, for example, about 10-1,000,000 monomers. As further examples, the PHB may comprise about 10-100,000 monomers, 100-100,000 monomers, 100-10,000 monomers, 500-5,000 monomers, 1,000-10,000 monomers, or 5,000-20,000 monomers. In a preferred embodiment, the PHB comprises about 100-12,000 monomers.

The molecular weight of PHB produced by the bacterium of the invention may be in the range of about 1,000-100,000,000 Da. For instance, the molecular weight of the PHB may be about 1,000-10,000 Da, 10,000-1,000,000 Da, 10,000-10,000,000 Da, or 10,000,000-100,000,000 Da. Preferably, the molecular weight of the PHB may be about 10,000-1,000,000 Da, such as 10,000-100,000 Da, 10,000-500,000 Da, 100,000-500,000 Da, 300,000-800,000 Da, or 500,000-1,000,000 Da.

PHB production is frequently referred to as a percentage of dry cellular weight. The microorganism of the invention may produce, for example, 0.005-0.995 wt % PHB. Preferably, the microorganism of the invention produces about 0.01 wt %, 0.1 wt %, 0.5 wt %, 1 wt %, 1.5 wt %, 2 wt %, 3 wt %, 5 wt %, 10 wt %, 20 wt %, 30 wt %, 40 wt %, 50 wt %, 60 wt %, 70 wt %, 80 wt %, 90 wt %, or 95 wt % PHB.

The physical characteristics of PHB are well known in the art. As a rough approximation, PHB has a Young's modulus of 1497-3500 MPa, a tensile strength of 18-43 MPa, elongation to break of 1.9-45%, a crystallinity of 60-80%, a melting temperature of 162-180° C., a crystallization temperature of 45-116° C., and/or a glass-transition temperature of −1.2-10° C.

Additionally, the microorganism of the invention may also produce or may be engineered to produce other products, such as ethanol (WO 2007/117157), acetate (WO 2007/117157), butanol (WO 2008/115080 and WO 2012/053905), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), isoprene (WO 2013/180584), fatty acids (WO 2013/191567), 2-butanol (WO 2013/185123), 1,2-propanediol (WO 2014/036152), 1-propanol (WO 2014/0369152), and chorismate-derived products (WO 2016/191625). In addition to one or more target products, the microorganism of the invention may also produce ethanol, acetate, and/or 2,3-butanediol. In certain embodiments, microbial biomass itself may be considered a product.

Preferably, the microorganism of the invention is not capable of degrading PHB. Organisms that natively produce PHB and other polyhydroxyalkanoates generally synthesize the polymers as a carbon storage material when other nutrients (e.g., nitrogen and phosphorous) are limiting and carbon is in excess. These organisms can then depolymerize/degrade the polymers when the limiting nutrient is replenished to have access to the stored carbon. For the purposes of maximizing PHB production, non-native producers, such as the microorganisms of the present invention, often have the advantage of not being capable of enzymatically degrading the polymers once they are produced. This essentially locks the carbon into the polymers permanently and can increase the yield.

“Selectivity” refers to the ratio of the production of a target product to the production of all fermentation products produced by a microorganism. The microorganism of the invention may be engineered to produce products at a certain selectivity or at a minimum selectivity. In one embodiment, a target product accounts for at least about 5%, 10%, 15%, 20%, 30%, 50%, or 75% of all fermentation products produced by the microorganism of the invention. In one embodiment, the target product accounts for at least 10% of all fermentation products produced by the microorganism of the invention, such that the microorganism of the invention has a selectivity for the target product of at least 10%. In another embodiment, the target product accounts for at least 30% of all fermentation products produced by the microorganism of the invention, such that the microorganism of the invention has a selectivity for the target product of at least 30%.

### Fermentation

The invention further provides a method of producing PHB comprising culturing the microorganism of the invention in the presence of a gaseous substrate, whereby the microorganism produces PHB. The gaseous substrate generally comprises one or more of CO, CO₂, and H₂.

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that comprises nutrients, vitamins, and/or minerals sufficient to permit growth of the microorganism. Preferably the aqueous culture medium is an anaerobic microbial growth medium, such as a minimal anaerobic microbial growth medium. Suitable media are well known in the art.

The culture should desirably be carried out under appropriate conditions for production of the target product. Typically, the culture is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition.

Operating a bioreactor at elevated pressures allows for an increased rate of gas mass transfer from the gas phase to the liquid phase. Accordingly, it may be preferable to perform the fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time, in turn, dictates the required volume of a bioreactor, the use of pressurized systems can greatly reduce the volume of the bioreactor required, and consequently the capital cost of the fermentation equipment.

In certain embodiments, the fermentation is performed in the absence of light or in the presence of an amount of light insufficient to meet the energetic requirements of photosynthetic or phototrophic microorganisms.

The methods of the invention may further involve separation or purification of PHB. The PHB may be separated or purified using any method known in the art. For example, cells may be collected by precipitation (Chen, Appl Microbiol Biotechnol, 57: 50-55, 2001) or continuous separation (Elbahloul, Appl Environ Microbiol, 75: 643-651, 2009; Heinrich, AMB Express, 2: 59, 2012) followed by lyophilization. Subsequent to freeze-drying, the polymer may be removed from cells with materials such as ethyl acetate (Chen, Appl Microbiol Biotechnol, 57: 50-55, 2001), acetone (Elbahloul, Appl Environ Microbiol, 75: 643-651, 2009), or sodium hypochlorite (Heinrich, AMB Express, 2: 59, 2012). The polymer may then be removed from residual/solubilized cell mass. A great number of alternative processes for purification of polyhydroxyalkanoates have been published and been developed but have not yet been established for larger-scale purification (Kunasundari, Express Polym Lett, 5, 620-634, 2011).

### EXAMPLES

The following examples further illustrate the invention but, of course, should not be construed to limit its scope in any way.

### Example 1

This example demonstrates construction of a Wood-Ljungdahl microorganism capable of PHB synthesis.

PHB pathway genes (phaC, phaA, and phaB) from C. necator (SEQ ID NOs: 1, 4, and 7) were introduced into C. autoethanogenum, a Wood-Ljungdahl microorganism that does not natively produce PHB. Of note, these species have significant differences in chromosomal GC-content. Specifically, C. necator has 66% GC-content (Pohlmann, Nat Biotechnol, 24: 1257-1262, 2006) and C. autoethanogenum has only 31% GC-content (Brown, Biotechnol Biofuels, 7: 40, 2014). Anticipating gene expression issues based on codon usage, the sequences of the PHB genes from C. necator were codon-adapted to better fit a higher expression profile for proteins in C. autoethanogenum. The genes, with novel sequences (SEQ ID NOs: 3, 6, and 9) coding for identical proteins as in C. necator, were synthesized and assembled into the expression vector pMTL83157 (SEQ ID NO: 10). This plasmid is similar to the pMTL8000 series (Heap, J Microbiol Methods, 78: 79-85, 2009) with a native Wood-Ljungdahl promoter taken from C. autoethanogenum to drive gene transcription. The genes were placed downstream of the promoter in the same order as they appear in the C. necator genome: phaC, phaA, and phaB. An antibiotic selection marker, catP, was also used. The resulting plasmid was named pPHB_01 (SEQ ID NO: 11) (FIG. 2).

pPHB_01 was inserted into C. autoethanogenum by bacterial conjugation using E. coli HB101, as described elsewhere (Mock, J Bacteriol, 197: 2965-2980, 2015). Separately, an “empty” pMTL83157 plasmid was inserted into C. autoethanogenum to serve as a negative control. These strains were then used to test PHB production from gaseous substrates.

In a preferred embodiment, the microorganism comprises enzyme that converts aceyl-CoA to acetoacetyl-CoA comprises an enzyme having at least 80% sequence identity to the amino acid sequence set forth in SEQ ID NO: 2, the enzyme that converts acetoacetyl-CoA to 3-hydroxybutyryl-CoA comprises an enzyme having at least 80% sequence identity to the amino acid sequence set forth in SEQ ID NO: 5, and/or the enzyme that converts 3-hydroxybutyryl-CoA to polyhydroxybutyrate comprises an enzyme having at least 80% sequence identity to the amino acid sequence set forth in SEQ ID NO: 8.

### Example 2

This example demonstrates the production of PHB from gaseous substrates in Schott bottles.

The strains constructed in Example 1 were grown in small batches to test for production of PHB. All work was conducted under strict anaerobic conditions (Hungate, Methods in microbiology, pages 117-132, Academic Press, New York, N.Y., 1969). Pressure-rated Schott bottles comprising modified PETC media (Köpke, Appl Environ Microbiol, 77: 5467-5475, 2011) with thiamphenicol for plasmid retention and 2-(N-morpholino) ethanesulfonic acid for buffering were inoculated with the strains, and gas comprising CO, CO₂, H₂, and N₂ (at 50, 18, 3, and 29%, respectively) as the sole carbon source was added to the bottles to 21 psi. The cultures were grown at 37° C. with rotary shaking.

Cell growth was monitored periodically until the cultures entered stationary phase. Upon completion of growth, the cells were no longer handled under anaerobic conditions. The cells were collected by centrifugation, their supernatants discarded, frozen at −20° C., and dried via lyophilization.

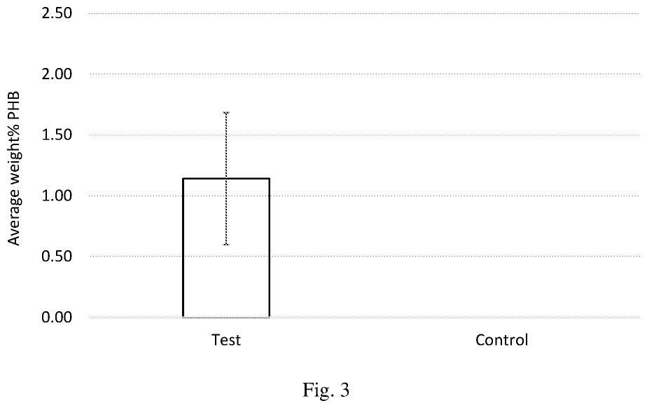

PHB yield was estimated by high-performance liquid chromatography (HPLC) in a similar manner as described elsewhere (Karr, Appl Environ Microbiol, 46, 1339-1344, 1983). Briefly, dried cells were treated with concentrated sulfuric acid and heated to convert the PHB to crotonic acid. Samples were cooled, diluted, filtered, and analyzed by HPLC with a UV-Vis detector to quantify the crotonic acid. The results of initial PHB production are summarized in FIG. 3, which shows the successful production of ˜1.15 wt % PHB in a Wood-Ljungdahl microorganism.

However, given the low yields compared to native producers such as Cupriavidus and Pseudomonas, which are capable of synthesizing polymers like PHB such that they account for upwards of 90% of their weight, it appears that PHB synthesis from gas in Wood-Ljungdahl microorganisms is not nearly as simple as in native producers growing on non-gaseous substrates. Without wishing to be bound by any particular theory, the inventors postulate that PHB production in Wood-Ljungdahl microorganisms may require codon-adaptation to overcome the differences in pH preference, oxygen requirements, substrate utilizations, etc. between Wood-Ljungdahl microorganisms and native PHB producers.

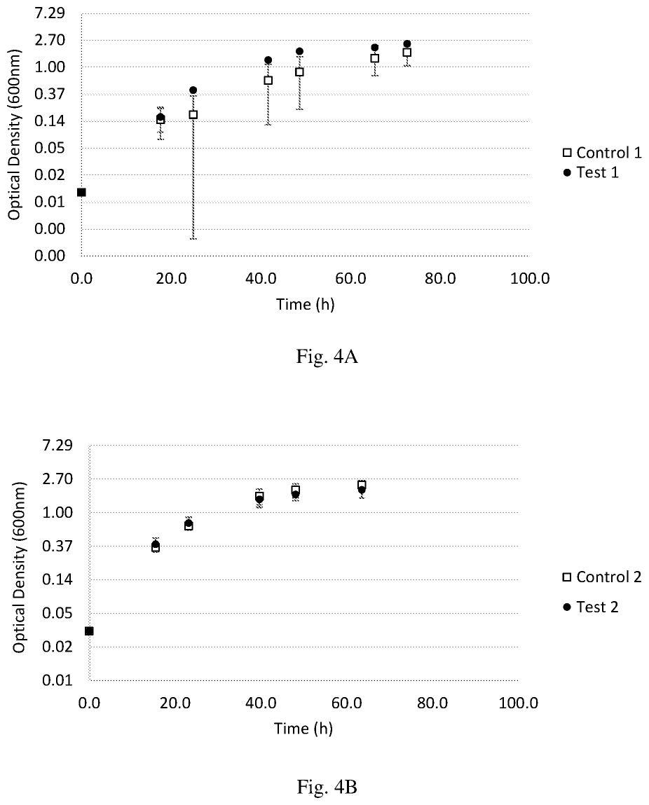

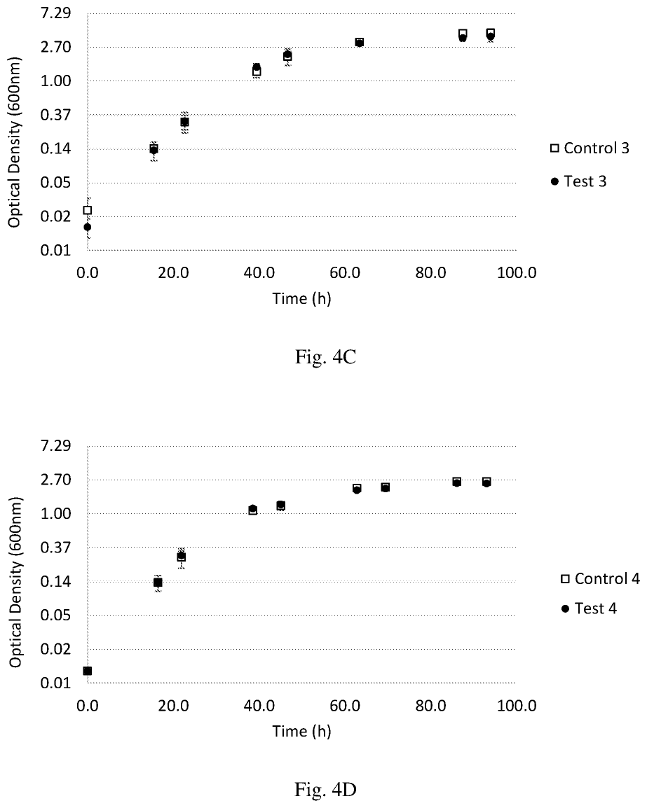

After achieving synthesis of PHB in C. autoethanogenum from gaseous substrates, the work described above was repeated with altered growth conditions in an effort to explore for conditions that may favor/improve PHB yield. In particular, experiments were performed to repeat the conditions described above (condition 1, FIG. 4A), to change the gas composition to 50/30/10/10 CO/CO₂/H₂/N₂ (condition 2, FIG. 4B), to extend the incubation of the culture into stationary phase (condition 3, FIG. 4C), and to periodically refresh the gas in the bottles (condition 4, FIG. 4D). As shown in FIGS. 4A-4D, growth was similar for both the engineered strain and the control strain under all tested conditions.

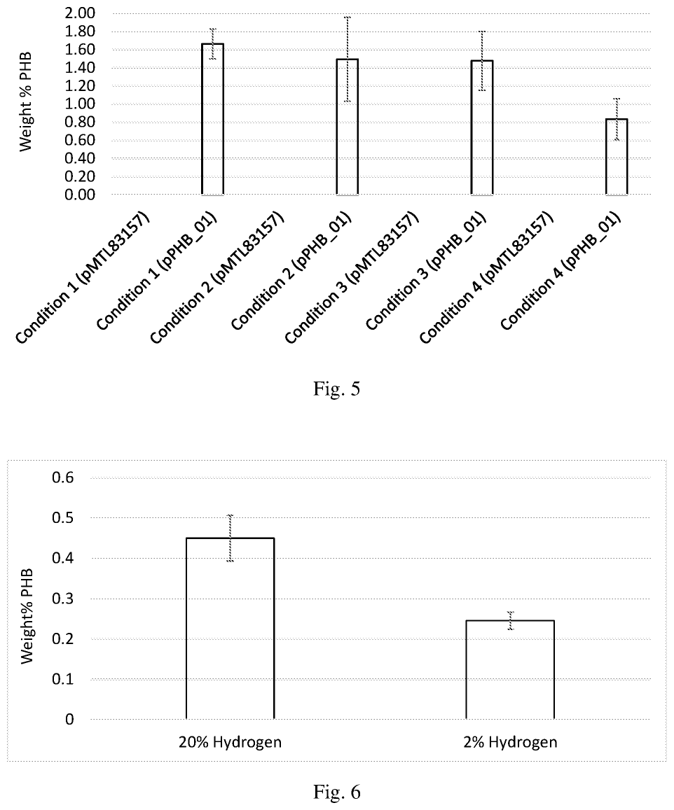

Cells were harvested and analyzed for PHB production as described above. The results are depicted in FIG. 5, which shows production of ˜1.65 wt % PHB under condition 1, ˜1.50 wt % PHB under condition 2, ˜1.50 wt % PHB under condition 3, and ˜0.85 wt % PHB under condition 4.

### Example 3

This example demonstrates the production of PHB from gaseous substrates in a continuous fermentation.

The strain constructed in Example 1 was tested under continuous fermentation using gas as the main source of carbon, under conditions similar to those described in Valgepea, Cell Syst, 4: 505-515, 2017. Similar to the experiments performed in Schott bottles, the continuous cultures were grown and handled anaerobically. Unlike the Schott bottles, the cultures were grown in a continuous fashion for approximately 20 days with constant feeding of media. Two different gas compositions were used for growth and PHB production: 50/20/20/10 CO/CO₂/H₂/Ar and 50/20/2/28 CO/CO₂/H₂/N₂. Gas uptake was monitored using mass spectrometry (MS) and samples were taken periodically to quantify liquid metabolites by HPLC.

PHB was not quantified until completion of the continuous fermentation. Similar to the Schott bottle experiments, cells were collected by centrifugation, frozen, and dried by lyophilization. Dried cells were subsequently analyzed for PHB by treatment with sulfuric acid and heat to convert the PHB to crotonic acid. PHB quantification was then carried out via HPLC. Results of PHB production in the continuous fermentation are shown in FIG. 6. In particular, microorganisms grown on 20% hydrogen gas produced ˜0.45 wt % PHB and microorganisms grown on 2% hydrogen gas produced ˜0.25 wt % PHB.

### Example 4

This example demonstrates fermenter optimization for increased PHB production.

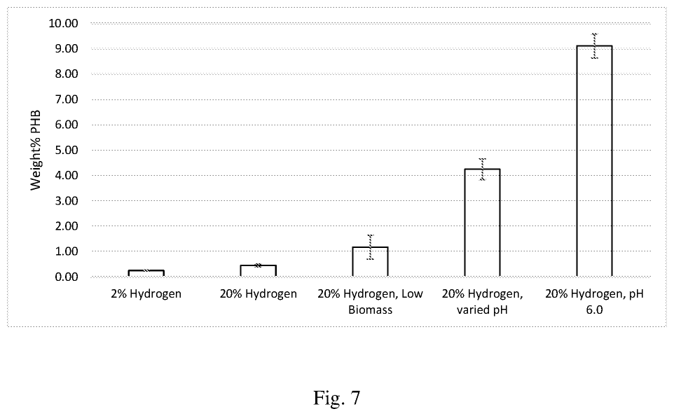

Various conditions were tested within continuous fermentations to increase the PHB content in cells. Pools of acetyl-CoA and NADPH increase at lower biomass concentrations (Valgepea, Cell Syst., 4: 505-515, 2017). Therefore, whether a lower steady-state biomass level would result in higher PHB through increased levels of the acetyl-CoA and NADPH pools was tested. Lowering the uptake rate of CO and, by extension, the biomass concentration in the fermenter, was shown to increase the flux of cellular resources toward PHB (FIG. 7).

Another factor found to increase PHB was pH. At higher pH, less acetic acid would diffuse and uncouple the proton motive force (PMF) (Valgepea, Cell Syst., 4: 505-515, 2017). Therefore, whether increasing the pH from 5 to 5.5 or 6 would drain less energy for maintaining the PMF was tested. The extra available energy would provide additional ATP to support PHB production by reducing acetate production needed for ATP production. Changing the pH from 5.0 to 5.5 or 6.0 resulted in increased PHB production (˜12.5 fold at pH 5.5). A pH value of 6.0, is difficult to maintain, however since C. autoethanogenum grows optimally at a more acidic pH.

### Example 5

This example demonstrates changes to transcriptional and metabolome level when producing PHB, as compared to the control (empty plasmid) strain.

Analysis of transcriptome data from RNA sequencing was based on a previously published R-script (Valgepea, Cell Syst., 4: 505-515, 2017) with the following modifications: use of the C. autoethanogenum NCBI reference sequence CP006763.1 and its annotated genome described in Brown, Biotechnol. Biofuels, 7: 40, 2014; addition of the nucleotide sequence for the three PHB genes (SEQ ID Nos: 3, 6, 9.)

A metabolomics package available in R (Livera and Bowne, R package, 2014) was used to perform the statistical analysis of the intracellular metabolomics data. This script normalizes and integrates the metabolomics data into a linear model fit (De Livera, Anal. Chem., 84: 10768-10776, 2012). Intracellular metabolite concentrations were normalised per biomass (μmol/gDCW) prior to importing the data into the script. A linear model fit using ordinary statistics (i.e. non-Bayesian) was used for the statistical analysis of the metabolome data (De Livera, Anal. Chem., 84: 10768-10776, 2012; De Livera, Metabolomics Tools for Natual Product Discovery, 2013).

Although arginine was not supplied, an upregulation was observed for the arginine deiminase pathway, an alternative route found to provide ATP in acetogens (Valgepea, Metab. Eng. 41: 202-211, 2017) (q-value <0.01): arginine deiminase (CAETHG_3021, ˜7 fold); ornithine carbamoyltransferase (CAETHG_3022, ˜6 fold); carbamate kinase (CAETHG_3025, ˜3.3 fold). Additionally, three genes encoding the Rnf complex, which is part of the energy conservation complex in acetogens (Schuchmann and Müller, Nat. Rev. Microbiol. 12: 809-821, 2014), showed an increase of ˜2 fold in the PHB strain: (CAETHG_3231, q-value=0.02; CAETHG_3228, q-value=0.04 and CAETHG_3230, q-value=0.03). These observations highlight changes in energy metabolism due to the heterologous production. In addition, expression of two genes of the Wood-Ljungdahl pathway (WLP) encoding for the CO dehydrogenase/acetyl-CoA synthase (CAETHG_1610, ˜1.4 fold; CAETHG_1611, ˜1.2 fold) and a gene encoding a (FeFe)-hydrogenase (CAETHG_1691, ˜2.5 fold) were upregulated in the PHB strain. These changes may reflect the increase needed for the production of acetyl-CoA and NADPH for PHB production (FIG. 1).

At the metabolome level, the PHB strain had a higher intracellular NADH/NAD⁺ ratio compared to the EP. This suggests potential changes in the redox state after PHB expression. Production of acetate, the main native by-product of C. autoethanogenum metabolism (Abrini, Arch. Microbiol., 161: 345-351, 1994; Marcellin, Green Chem., 18: 3020-3028, 2016), decreased compared to the EP strain on syngas (p-value<0.01; two-tailed equal variance t-test). No change was observed on steel mill off gas. In contrast to E. coli, in C. autoethanogenum the NADPH pool was found to be larger than the NADH pool, with a ratio of 2.2:1 NADPH+H⁺ and NADP to NADH+H⁺ and NADH, respectively 36.8:1 NADPH+H⁺ to NADH+H⁺ demonstrating the driving force of NADPH in acetogenic carboxydotrophic Clostridia with CO as substrate.

### Example 6

This example shows results of genome-scale metabolic model reconstructions (GEM). The genome-scale metabolic model GEM iCLAU786 (Valgepea, Cell Syst., 4: 505-515, 2017) was used with the addition of the PHB pathway. Simulations were performed for the PHB strain grown on syngas in all conditions listed above.

Flux simulations confirmed that less CO₂ was dissipated in the conditions with higher PHB (i.e. “Low biomass” and “pH5.5”). Additionally, as observed previously (Valgepea, Cell Syst., 4: 505-515, 2017), these simulations also showed that CO₂ was directly reduced to formate by H₂ through the formate-H₂ lyase activity of the electron-bifurcating hydrogenase-formate dehydrogenase (HytA-E/FdhA) enzyme complex (Wang, J. Bacteriol., 195: 4373-4386, 2013). This offers an advantage over the reduction of CO₂ by the redox-consuming formate dehydrogenase because no redox is consumed during CO₂ reduction in the WLP using the former enzyme complex. It was also observed that in the “Low biomass” and “pH5.5” experiments, balancing the total amount of reduced ferredoxin was achieved by either increasing or decreasing the flux to some key reactions, like the AOR (Aldehyde ferredoxin oxidoreductase), Nfn complex, or methylene THF reductase bifurcating reaction, compared to the control (PHB20).

Surprisingly, the “control” condition (PHB20) had, in silico, lower maintenance ATP cost (mmol/gDCW/h), and maintenance ATP costs from total ATP production (mATP %) compared to the “PHBpH5.5” condition.

Simulations to determine if ATP, NADH, NADPH or reduced ferredoxin (Fd_{red}) was limiting PHB production were also run. The simulations showed that when ATP was provided, PHB production (mmol/gDCW/h) reached its maximum value among the “limiting” candidates in all conditions tested (i.e. “PHB20,” “PHBLowBiomass,” and “PHBpH5.5.”) This observation is consistent with the understanding of acetogen metabolism being ATP-limited (Schuchmann and Müller, Nat. Rev. Microbiol. 12: 809-821, 2014). The model also showed that followed by the ATP limitation, PHB production is limited by Fd_{red}, NADPH, and then NADH availability (FIG. 8).

This result confirms the importance of ATP and Fd_{red} as high energy carriers in acetogens. As ATP mostly supports anabolism and cellular maintenance, Fd_{red} is essential for the Rnf energy conservation complex (Biegel, Cell. Mol. Life Sci. 68: 613-634, 2011) and only Fd_{red} is known to provide electrons for the reduction of CO₂ to CO in the carbonyl branch of the WLP (Schuchmann and Müller, Nat. Rev. Microbiol. 12: 809-821, 2014).

One embodiment is a method of controlling polyhydroxyalkanoate production comprising culturing a recombinant C1-fixing microorganism capable of producing polyhydroxyalkanoate in the presence of one or more gaseous substrates, whereby the microorganism produces polyhydroxyalkanoate; and wherein polyhydroxyalkanoate production is controlled by one or more cofactors selected from ATP, NADH, NADPH, reduced ferredoxin (Fd_{red}), or any combination thereof.

The method of an embodiment, further comprising the utilization of NADPH over ATP, NADH, or FD_{red}, is increased relative to the parental microorganism.

The method of an embodiment, further comprising the utilization of NADH over ATP, NADPH, or FD_{red}, is increased relative to the parental microorganism.

The method of an embodiment, further comprising limiting the utilization of one or more cofactors selected from ATP, NADH, NADPH, reduced ferredoxin (Fd_{red}), or any combination thereof.

The method of an embodiment, further comprising a ratio of NADPH+H⁺ and NADP to NADH+H⁺ and NADH at about 2.2:1.

The method of an embodiment, further comprising a ratio of NADPH+H⁺ to NADH+H⁺ at about 36.8:1.

The method of an embodiment, further comprising the polyhydroxyalkanoate is used in a plastic product.

The method of an embodiment, wherein the plastic product has a reduced environmental impact as compared to a petroleum derived plastic product.

The method of an embodiment, wherein the polyhydroxyalkanoate is polyhydroxybutyrate (PHB).

The method of an embodiment, further comprising the microorganism producing polyhydroxyalkanoate and lactate.

The method of an embodiment, wherein the recombinant C1-fixing microorganism comprises: a. a nucleic acid encoding a heterologous acetyl-CoA C-acetyltransferase (EC 2.3.1.9) enzyme, b. a nucleic acid encoding a heterologous acetoacetyl-CoA reductase (EC 1.1.1.36), a nucleic acid encoding a heterologous 3-hydroxybutyryl-CoA hydratase (E.C. 4.2.1.119), a nucleic acid encoding a heterologous acetoacetyl-CoA hydratase (EC 4.2.1.119), or a nucleic acid encoding a heterologous 3-hydroxybutyryl-CoA dehydrogenase (EC 1.1.1.157) enzyme, and c. a nucleic acid encoding a heterologous polyhydroxyalkanoate synthase (EC 2.3.1.-) enzyme.

The method of an embodiment, wherein the acetoacetyl-CoA reductase is phaB (EC 1.1.1.36).

The method of an embodiment, wherein the acetyl-CoA C-acetyltransferase is derived from a parental microorganism selected from the group consisting of Acinetobacter baumannii, Aeromonas hydrophilia, Alcaligenes latus, Arthrospira platensis, Bacillus subtilis, Burkholderia cepacia, Clostridium acetobutylicum, Cupriavidus necator, Escherichia coli, Haloferax mediterranei, Pseudomonas aeruginosa, Pseudomonas fluorescens, Pseudomonas mandelii, Pseudomonas oleovorans, Pseudomonas putida, and Streptomyces coelicolor.

The method of an embodiment, wherein the acetoacetyl-CoA reductase is derived from a parental microorganism selected from the group consisting of Acinetobacter baumannii, Aeromonas hydrophilia, Alcaligenes latus, Arthrospira platensis, Bacillus subtilis, Burkholderia cepacia, Cupriavidus necator, Haloferax mediterranei, Pseudomonas aeruginosa, Pseudomonas fluorescens, Pseudomonas mandelii, Pseudomonas oleovorans, Pseudomonas putida, and Streptomyces coelicolor.

The method of an embodiment, wherein the 3-hydroxybutyryl-CoA dehydrogenase is derived from Clostridium beijerinckii, Clostridium acetobutylicum, or Clostridium kluyveri.

The method of an embodiment, wherein the polyhydroxyalkanoate synthase is derived from a parental microorganism selected from the group consisting of Acinetobacter baumannii, Aeromonas caviae, Aeromonas hydrophilia, Alcaligenes latus, Arthrospira platensis, Bacillus subtilis, Burkholderia cepacia, Cupriavidus necator, Haloferax mediterranei, Pseudomonas aeruginosa, Pseudomonas fluorescens, Pseudomonas mandelii, Pseudomonas oleovorans, Pseudomonas putida, Pseudomonas sp. 61-3, Rhodospirillum rubrum, and Streptomyces coelicolor.

The method of an embodiment, wherein the microorganism is a member of a genus selected from the group consisting of Acetobacterium, Alkalibaculum, Blautia, Butyribacterium, Clostridium, Eubacterium, Moorella, Oxobacter, Sporomusa, and Thermoanaerobacter.

The method of an embodiment, wherein the microorganism is derived from a parental microorganism selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi.

The method of an embodiment, wherein the microorganism is derived from a parental bacterium selected from the group consisting of Clostridium autoethanogenum, Clostridium coskatii, Clostridium ljungdahlii, and Clostridium ragsdalei.

The method of an embodiment, wherein the one or more gaseous substrates comprising one or more of CO, CO₂, and H₂.

The method of an embodiment, wherein the microorganism is anaerobic.

The method of an embodiment, wherein the microorganism is not capable of degrading polyhydroxybutyrate.

The method of an embodiment, wherein the microorganism is not phototrophic, photosynthetic, or methanotrophic.

The method of an embodiment, wherein the culturing is performed under anaerobic conditions.

The method of an embodiment, wherein the culturing is performed in the absence of carbohydrate substrates.

The method of an embodiment, wherein the culturing is performed in the absence of light.

One embodiment provides a recombinant C1-fixing microorganism capable of producing polyhydroxyalkanoate comprising: a. a nucleic acid encoding a heterologous acetyl-CoA C-acetyltransferase (EC 2.3.1.9) enzyme, b. a nucleic acid encoding a heterologous acetoacetyl-CoA reductase (EC 1.1.1.36), a nucleic acid encoding a heterologous 3-hydroxybutyryl-CoA hydratase (E.C. 4.2.1.119), a nucleic acid encoding a heterologous acetoacetyl-CoA hydratase (EC 4.2.1.119), or a nucleic acid encoding a heterologous 3-hydroxybutyryl-CoA dehydrogenase (EC 1.1.1.157) enzyme, and c. a nucleic acid encoding a heterologous polyhydroxyalkanoate synthase (EC 2.3.1.-) enzyme, wherein the microorganism is capable of increased utilization of a co-factor selected from ATP, NADH, NADPH, reduced ferredoxin (FD_{red}), or any combination thereof relative to the parental microorganism.

Another embodiment provides a method of producing polyhydroxyalkanoate comprising culturing the microorganism in the presence of a gaseous substrate, whereby the microorganism produces polyhydryoxyalkanoate.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means ±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of controlling polyhydroxyalkanoate production comprising culturing a recombinant C1-fixing microorganism capable of producing polyhydroxyalkanoate in the presence of one or more gaseous substrates, whereby the microorganism produces polyhydroxyalkanoate; and wherein polyhydroxyalkanoate production is controlled by one or more cofactors selected from ATP, NADH, NADPH, reduced ferredoxin (Fd_{red}), or any combination thereof.

2. The method of claim 1, further comprising the utilization of NADPH over ATP, NADH, or FD_{red}, is increased relative to the parental microorganism.

3. The method of claim 1, further comprising the utilization of NADH over ATP, NADPH, or FD_{red}, is increased relative to the parental microorganism.

4. The method of claim 1, further comprising limiting the utilization of one or more cofactors selected from ATP, NADH, NADPH, reduced ferredoxin (Fd_{red}), or any combination thereof.

5. The method of claim 1, further comprising a ratio of NADPH+H⁺ and NADP to NADH+H⁺ and NADH at about 2.2:1.

6. The method of claim 1, further comprising a ratio of NADPH+H⁺ to NADH+H⁺ at about 36.8:1.

7. The method of claim 1, further comprising the polyhydroxyalkanoate is used in a plastic product.

8. The method of claim 7, wherein the plastic product has a reduced environmental impact as compared to a petroleum derived plastic product.

9. The method of claim 1, wherein the polyhydroxyalkanoate is polyhydroxybutyrate (PHB).

10. The method of claim 1, further comprising the recombinant C1-fixing microorganism producing polyhydroxyalkanoate and lactate.

11. The method of claim 1, wherein the recombinant C1-fixing microorganism comprises:
a. a nucleic acid encoding a heterologous acetyl-CoA C-acetyltransferase (EC 2.3.1.9) enzyme,
b. a nucleic acid encoding a heterologous acetoacetyl-CoA reductase (EC 1.1.1.36), a nucleic acid encoding a heterologous 3-hydroxybutyryl-CoA hydratase (E.C. 4.2.1.119), a nucleic acid encoding a heterologous acetoacetyl-CoA hydratase (EC 4.2.1.119), or a nucleic acid encoding a heterologous 3-hydroxybutyryl-CoA dehydrogenase (EC 1.1.1.157) enzyme, and
c. a nucleic acid encoding a heterologous polyhydroxyalkanoate synthase (EC 2.3.1.-) enzyme.

12. The method of claim 11, wherein the acetoacetyl-CoA reductase is phaB (EC 1.1.1.36).

13. The method of claim 11, wherein the acetoacetyl-CoA reductase is derived from a parental microorganism selected from the group consisting of Acinetobacter baumannii, Aeromonas hydrophilia, Alcaligenes latus, Arthrospira platensis, Bacillus subtilis, Burkholderia cepacia, Cupriavidus necator, Haloferax mediterranei, Pseudomonas aeruginosa, Pseudomonas fluorescens, Pseudomonas mandelii, Pseudomonas oleovorans, Pseudomonas putida, and Streptomyces coelicolor.

14. The method of claim 1, wherein the microorganism is derived from a parental microorganism selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kiuvi.

15. The method of claim 1, wherein the microorganism is derived from a parental bacterium selected from the group consisting of Clostridium autoethanogenum, Clostridium coskatii, Clostridium ljungdahlii, and Clostridium ragsdalei.

16. The method of claim 1, wherein the one or more gaseous substrates comprising one or more of CO, CO₂, and H₂.

17. The method of claim 1, wherein the microorganism is anaerobic.

18. The method of claim 9, wherein the microorganism is not capable of degrading polyhydroxybutyrate.

19. A recombinant C1-fixing microorganism capable of producing polyhydroxyalkanoate comprising: a. a nucleic acid encoding a heterologous acetyl-CoA C-acetyltransferase (EC 2.3.1.9) enzyme,
b. a nucleic acid encoding a heterologous acetoacetyl-CoA reductase (EC 1.1.1.36), a nucleic acid encoding a heterologous 3-hydroxybutyryl-CoA hydratase (E.C. 4.2.1.119), a nucleic acid encoding a heterologous acetoacetyl-CoA hydratase (EC 4.2.1.119), or a nucleic acid encoding a heterologous 3-hydroxybutyryl-CoA dehydrogenase (EC 1.1.1.157) enzyme, and
c. a nucleic acid encoding a heterologous polyhydroxyalkanoate synthase (EC 2.3.1.-) enzyme, wherein the microorganism is capable of increased utilization of a co-factor selected from ATP, NADH, NADPH, reduced ferredoxin (FD_{red}), or any combination thereof relative to the parental microorganism.

20. A method of producing polyhydroxyalkanoate comprising culturing the microorganism of claim 19 in the presence of a gaseous substrate, whereby the microorganism produces polyhydryoxyalkanoate.


SEQUENCE LISTING (11 sequences)

SEQ ID NO: 1 - DNA, 1179 bp, Cupriavidus necator
atgactgacgttgtcatcgtatccgccgcccgcaccgcggtcggcaagtttggcggctcgctggccaagatcccggcaccggaactgggtgccgtggtcatcaaggccgcgctggagcgcgccggcgtcaagccggagcaggtgagcgaagtcatcatgggccaggtgctgaccgccggttcgggccagaaccccgcacgccaggccgcgatcaaggccggcctgccggcgatggtgccggccatgaccatcaacaaggtgtgcggctcgggcctgaaggccgtgatgctggccgccaacgcgatcatggcgggcgacgccgagatcgtggtggccggcggccaggaaaacatgagcgccgccccgcacgtgctgccgggctcgcgcgatggtttccgcatgggcgatgccaagctggtcgacaccatgatcgtcgacggcctgtgggacgtgtacaaccagtaccacatgggcatcaccgccgagaacgtggccaaggaatacggcatcacacgcgaggcgcaggatgagttcgccgtcggctcgcagaacaaggccgaagccgcgcagaaggccggcaagtttgacgaagagatcgtcccggtgctgatcccgcagcgcaagggcgacccggtggccttcaagaccgacgagttcgtgcgccagggcgccacgctggacagcatgtccggcctcaagcccgccttcgacaaggccggcacggtgaccgcggccaacgcctcgggcctgaacgacggcgccgccgcggtggtggtgatgtcggcggccaaggccaaggaactgggcctgaccccgctggccacgatcaagagctatgccaacgccggtgtcgatcccaaggtgatgggcatgggcccggtgccggcctccaagcgcgccctgtcgcgcgccgagtggaccccgcaagacctggacctgatggagatcaacgaggcctttgccgcgcaggcgctggcggtgcaccagcagatgggctgggacacctccaaggtcaatgtgaacggcggcgccatcgccatcggccacccgatcggcgcgtcgggctgccgtatcctggtgacgctgctgcacgagatgaagcgccgtgacgcgaagaagggcctggcctcgctgtgcatcggcggcggcatgggcgtggcgctggcagtcgagcgcaaa

SEQ ID NO: 2 - protein, 393 aa, Cupriavidus necator
MTDVVIVSAARTAVGKFGGSLAKIPAPELGAVVIKAALERAGVKPEQVSEVIMGQVLTAGSGQNPARQAAIKAGLPAMVPAMTINKVCGSGLKAVMLAANAIMAGDAEIVVAGGQENMSAAPHVLPGSRDGFRMGDAKLVDTMIVDGLWDVYNQYHMGITAENVAKEYGITREAQDEFAVGSQNKAEAAQKAGKFDEEIVPVLIPQRKGDPVAFKTDEFVRQGATLDSMSGLKPAFDKAGTVTAANASGLNDGAAAVVVMSAAKAKELGLTPLATIKSYANAGVDPKVMGMGPVPASKRALSRAEWTPQDLDLMEINEAFAAQALAVHQQMGWDTSKVNVNGGAIAIGHPIGASGCRILVTLLHEMKRRDAKKGLASLCIGGGMGVALAVERK

SEQ ID NO: 3 - DNA, 1182 bp, Artificial sequence
  Synthetic nucleic acid
atgactgatgtagtaatagtatctgcagcaagaacggcagttggaaaatttggaggatctttggctaaaatacctgcaccagaactaggggcagtggttataaaagcagcactggaaagggccggcgtcaaaccagaacaggtttcagaagtaattatgggacaagttttaacagctggatcaggtcagaatcctgcaagacaagcagctattaaagcaggacttccagcaatggtgccagctatgaccataaataaggtttgtggcagtggattaaaggcagtaatgttggcagctaatgcaataatggcaggtgatgcagaaatagttgtagcaggtggtcaagaaaatatgtctgctgcaccacatgtactgccgggatctagggatggttttaggatgggagatgcaaaattggtggatacaatgatagtagatggactttgggatgtatacaatcagtatcacatgggaattacagctgaaaatgttgctaaagaatatggaataactagagaagctcaagatgagtttgcagtaggttcacaaaataaggctgaagcagcacaaaaagccggaaaatttgatgaggaaatagttcctgtattaataccacaaagaaaaggagatcctgtagcatttaaaacagatgaatttgtacgccagggagctacattggattcaatgtctggcttaaaaccggcctttgataaggcaggtactgtaactgcagctaatgcaagtgggttaaatgatggagcagcagcagttgtggtaatgtcagcagctaaagctaaagagttgggtcttactccacttgcaactataaagagctatgcaaatgcaggagtcgatccaaaggtcatgggcatgggtcctgttccagcgtctaaaagagcactaagtagagctgaatggacaccacaagacctggatcttatggaaataaatgaagcatttgctgcgcaggcccttgctgtccatcaacaaatgggatgggatacttcaaaagtaaatgtgaatggaggagctattgccatagggcatcccattggagccagtggatgccgcattttagtaactttacttcatgagatgaaaagaagagatgcaaaaaaaggacttgccagcttatgtataggtggaggaatgggtgtagctttagccgttgaaagaaaataa

SEQ ID NO: 4 - DNA, 741 bp, Cupriavidus necator
atgactcagcgcattgcgtatgtgaccggcggcatgggtggtatcggaaccgccatttgccagcggctggccaaggatggctttcgtgtggtggccggttgcggccccaactcgccgcgccgcgaaaagtggctggagcagcagaaggccctgggcttcgatttcattgcctcggaaggcaatgtggctgactgggactcgaccaagaccgcattcgacaaggtcaagtccgaggtcggcgaggttgatgtgctgatcaacaacgccggtatcacccgcgacgtggtgttccgcaagatgacccgcgccgactgggatgcggtgatcgacaccaacctgacctcgctgttcaacgtcaccaagcaggtgatcgacggcatggccgaccgtggctggggccgcatcgtcaacatctcgtcggtgaacgggcagaagggccagttcggccagaccaactactccaccgccaaggccggcctgcatggcttcaccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 5 - protein, 246 aa, Cupriavidus necator
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 6 - DNA, 741 bp, Artificial sequence
  Synthetic nucleic acid
atgacacagagaatagcttatgtaactggaggaatggggggaattggcacggcaatatgtcagagattagcaaaggatggttttagagtagttgcgggttgtggcccaaactcaccgaggagagaaaaatggttggaacagcagaaagctctcggatttgactttatagctagtgagggtaatgttgctgattgggattcaacaaagacagcttttgataaggttaagtcagaagtgggtgaagtagatgtgctcataaataatgctgggatcacaagagatgtagtttttagaaaaatgacaagagctgactgggatgctgtaatagatacaaatcttactagcttattcaatgtaacgaaacaggttatagatggaatggcagataggggatggggtaggatagtaaatatttcatcagtaaatggtcaaaaaggacaatttggacaaacaaattattcaactgccaaggcaggacttcatggatttacgatggcacttgcacaggaagtagctactaaaggagttactgtaaatacagtttctccaggatacatagctactgatatggtaaaagctattaggcaggatgtattagataagattgtagcaacaatacctgtgaagagacttggcttacctgaagaaatagcatcaatatgtgcttggttatccagtgaagaatcaggattttctacaggagctgatttctccttgaatggtggacttcacatgggataa

SEQ ID NO: 7 - DNA, 1770 bp, Cupriavidus necator
atggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgcccaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatga

SEQ ID NO: 8 - protein, 588 aa, Cupriavidus necator
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAK

SEQ ID NO: 9 - DNA, 1767 bp, Artificial sequence
  Synthetic nucleic acid
atggctacaggaaaaggagcagcagctagtacacaggaaggaaaatcacaaccgtttaaagttacaccaggtccttttgacccagccacctggttagaatggtcacgccagtggcagggtactgagggaaatggacatgcagcagcaagtggcattccaggattggatgctcttgctggggtaaagatagctccagctcagcttggagatatacaacagagatatatgaaagatttttcagctctttggcaagccatggcagaaggaaaagcagaagcgacaggtccacttcatgacagaagatttgctggagatgcttggagaactaatttgccatatagatttgctgccgctttttatcttttgaatgccagagctttaactgagcttgcagatgctgtagaagctgatgctaaaacaagacagagaataagatttgctatttcccaatgggttgatgctatgtcgccggcgaattttttagctacaaatcctgaggcacagagattacttattgagtccggaggagaatcactaagggctggagtgagaaatatgatggaagatttgaccagaggaaagatctctcagacagacgaatctgcatttgaagtaggaaggaatgttgcggtaactgaaggtgctgttgtatttgaaaatgaatattttcaattactacaatataaacctttgacagacaaggtgcatgctagacctcttcttatggttccaccttgtataaataaatattatattttagatcttcagcctgaatcatctttagtaagacacgttgtagaacaaggtcacacagtatttttagtttcttggagaaatcctgacgcatctatggccggcagtacctgggatgattatatagaacatgcagcaataagggcaatagaagttgctagagatataagcggtcaggacaagattaatgtacttggattttgtgtaggaggaactatagtttcaacagcactggcagtattggctgctagaggagaacatccagcagcatcggtaactttacttacaacacttttagattttgcagatactggaatattagacgtatttgttgatgaaggacacgtacaattaagagaggcaaccttgggtggaggagctggtgcaccctgtgctcttcttagagggttagaattagcaaatacttttagttttttacgacctaatgacttagtttggaattatgtagtggataattaccttaaaggaaacacacctgtaccttttgatttattattttggaatggagatgcaaccaacttgcctggtccttggtattgttggtatcttagacatacctaccttcaaaatgaattaaaagttcctggcaaactcactgtatgcggtgtccccgttgatttggcttcaatagatgtaccaacttatatatacggaagtagagaggatcatatcgtaccttggactgcggcttacgcttctacagcattgctggctaataaattgagatttgtattaggagcttctggtcacatagctggagtaataaatccaccagccaaaaataaacgaagtcattggaccaatgatgcacttcctgaatctccacagcagtggcttgctggtgcaatagaacatcatggttcatggtggcccgactggacagcatggttggcaggtcaagccggtgctaaaagggctgcgccagcaaactatggcaatgcaagatacagggctatagaaccggctccaggaagatacgttaaagcaaaataa

SEQ ID NO: 10 - DNA, 5024 bp, Artificial sequence
  Synthetic nucleic acid
cctgcaggataaaaaaattgtagataaattttataaaatagttttatctacaatttttttatcaggaaacagctatgaccgcggccgcagatagtcataatagttccagaatagttcaatttagaaattagactaaacttcaaaatgtttgttaaatatataccaaactagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcaattgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaatagggttttaggtaaaattaagttattttaagaagtaattacaataaaaattgaagttattgctttaaggagggaattattcatatgaccatgattacgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 11 - DNA, 8765 bp, Artificial sequence
  Synthetic nucleic acid
cctgcaggataaaaaaattgtagataaattttataaaatagttttatctacaatttttttatcaggaaacagctatgaccgcggccgcagatagtcataatagttccagaatagttcaatttagaaattagactaaacttcaaaatgtttgttaaatatataccaaactagtatagatattttttaaatactggacttaaacagtagtaatttgcctaaaaaattttttcaattttttttaaaaaatccttttcaagttgtacattgttatggtaatatgtaattgaagaagttatgtagtaatattgtaaacgtttcttgatttttttacatccatgtagtgcttaaaaaaccaaaatatgtcacatgcaattgtatatttcaaataacaatatttattttctcgttaaattcacaaataatttattaataatatcaataaccaagattatacttaaatggatgtttattttttaacacttttatagtaaatatatttattttatgtagtaaaaaggttataattataattgtatttattacaattaattaaaataaaaatagggttttaggtaaaattaagttattttaagaagtaattacaataaaaattgaagttattgctttaaggagggaattattcatatggctacaggaaaaggagcagcagctagtacacaggaaggaaaatcacaaccgtttaaagttacaccaggtccttttgacccagccacctggttagaatggtcacgccagtggcagggtactgagggaaatggacatgcagcagcaagtggcattccaggattggatgctcttgctggggtaaagatagctccagctcagcttggagatatacaacagagatatatgaaagatttttcagctctttggcaagccatggcagaaggaaaagcagaagcgacaggtccacttcatgacagaagatttgctggagatgcttggagaactaatttgccatatagatttgctgccgctttttatcttttgaatgccagagctttaactgagcttgcagatgctgtagaagctgatgctaaaacaagacagagaataagatttgctatttcccaatgggttgatgctatgtcgccggcgaattttttagctacaaatcctgaggcacagagattacttattgagtccggaggagaatcactaagggctggagtgagaaatatgatggaagatttgaccagaggaaagatctctcagacagacgaatctgcatttgaagtaggaaggaatgttgcggtaactgaaggtgctgttgtatttgaaaatgaatattttcaattactacaatataaacctttgacagacaaggtgcatgctagacctcttcttatggttccaccttgtataaataaatattatattttagatcttcagcctgaatcatctttagtaagacacgttgtagaacaaggtcacacagtatttttagtttcttggagaaatcctgacgcatctatggccggcagtacctgggatgattatatagaacatgcagcaataagggcaatagaagttgctagagatataagcggtcaggacaagattaatgtacttggattttgtgtaggaggaactatagtttcaacagcactggcagtattggctgctagaggagaacatccagcagcatcggtaactttacttacaacacttttagattttgcagatactggaatattagacgtatttgttgatgaaggacacgtacaattaagagaggcaaccttgggtggaggagctggtgcaccctgtgctcttcttagagggttagaattagcaaatacttttagttttttacgacctaatgacttagtttggaattatgtagtggataattaccttaaaggaaacacacctgtaccttttgatttattattttggaatggagatgcaaccaacttgcctggtccttggtattgttggtatcttagacatacctaccttcaaaatgaattaaaagttcctggcaaactcactgtatgcggtgtccccgttgatttggcttcaatagatgtaccaacttatatatacggaagtagagaggatcatatcgtaccttggactgcggcttacgcttctacagcattgctggctaataaattgagatttgtattaggagcttctggtcacatagctggagtaataaatccaccagccaaaaataaacgaagtcattggaccaatgatgcacttcctgaatctccacagcagtggcttgctggtgcaatagaacatcatggttcatggtggcccgactggacagcatggttggcaggtcaagccggtgctaaaagggctgcgccagcaaactatggcaatgcaagatacagggctatagaaccggctccaggaagatacgttaaagcaaaataagaattcgagctcggtaccaggaggatattaaaatgactgatgtagtaatagtatctgcagcaagaacggcagttggaaaatttggaggatctttggctaaaatacctgcaccagaactaggggcagtggttataaaagcagcactggaaagggccggcgtcaaaccagaacaggtttcagaagtaattatgggacaagttttaacagctggatcaggtcagaatcctgcaagacaagcagctattaaagcaggacttccagcaatggtgccagctatgaccataaataaggtttgtggcagtggattaaaggcagtaatgttggcagctaatgcaataatggcaggtgatgcagaaatagttgtagcaggtggtcaagaaaatatgtctgctgcaccacatgtactgccgggatctagggatggttttaggatgggagatgcaaaattggtggatacaatgatagtagatggactttgggatgtatacaatcagtatcacatgggaattacagctgaaaatgttgctaaagaatatggaataactagagaagctcaagatgagtttgcagtaggttcacaaaataaggctgaagcagcacaaaaagccggaaaatttgatgaggaaatagttcctgtattaataccacaaagaaaaggagatcctgtagcatttaaaacagatgaatttgtacgccagggagctacattggattcaatgtctggcttaaaaccggcctttgataaggcaggtactgtaactgcagctaatgcaagtgggttaaatgatggagcagcagcagttgtggtaatgtcagcagctaaagctaaagagttgggtcttactccacttgcaactataaagagctatgcaaatgcaggagtcgatccaaaggtcatgggcatgggtcctgttccagcgtctaaaagagcactaagtagagctgaatggacaccacaagacctggatcttatggaaataaatgaagcatttgctgcgcaggcccttgctgtccatcaacaaatgggatgggatacttcaaaagtaaatgtgaatggaggagctattgccatagggcatcccattggagccagtggatgccgcattttagtaactttacttcatgagatgaaaagaagagatgcaaaaaaaggacttgccagcttatgtataggtggaggaatgggtgtagctttagccgttgaaagaaaataaatcattctgaattcgagctcggtaggaggtcagaatgacacagagaatagcttatgtaactggaggaatggggggaattggcacggcaatatgtcagagattagcaaaggatggttttagagtagttgcgggttgtggcccaaactcaccgaggagagaaaaatggttggaacagcagaaagctctcggatttgactttatagctagtgagggtaatgttgctgattgggattcaacaaagacagcttttgataaggttaagtcagaagtgggtgaagtagatgtgctcataaataatgctgggatcacaagagatgtagtttttagaaaaatgacaagagctgactgggatgctgtaatagatacaaatcttactagcttattcaatgtaacgaaacaggttatagatggaatggcagataggggatggggtaggatagtaaatatttcatcagtaaatggtcaaaaaggacaatttggacaaacaaattattcaactgccaaggcaggacttcatggatttacgatggcacttgcacaggaagtagctactaaaggagttactgtaaatacagtttctccaggatacatagctactgatatggtaaaagctattaggcaggatgtattagataagattgtagcaacaatacctgtgaagagacttggcttacctgaagaaatagcatcaatatgtgcttggttatccagtgaagaatcaggattttctacaggagctgatttctccttgaatggtggacttcacatgggataaaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc